(19) United States
(12) Patent Application Publication
(10) Pub. No.: US 2024/0189307 A1
(43) Pub. Date: Jun. 13, 2024

(54) METHODS OF STABILIZING THE NEURONAL PROTEOME AGAINST COLLAPSE AND PROTECTING VASCULAR CELLS

(71) Applicant: ALBERT EINSTEIN COLLEGE OF MEDICINE, Bronx, NY (US)

(72) Inventors: Ana Maria CUERVO, Bronx, NY (US); Evripidis GAVATHIOTIS, Flushing, NY (US)

(21) Appl. No.: 18/556,140

(22) PCT Filed: Apr. 21, 2022
(86) PCT No.: PCT/US2022/025757

§ 371 Date: Oct. 19, 2023

Related U.S. Application Data
(60) Provisional application No. 63/177,504, filed on Apr. 21, 2021.
(60) Provisional application No. 63/323,942, filed on Mar. 25, 2022.

(51) Int. Cl. A61K 31/498; A61P 9/10; A61P 25/28

(52) U.S. Cl. CPC A61K 31/498; A61P 9/10; A61P 25/28

(57) ABSTRACT

The disclosure provides method of enhancing neuronal, vascular cell, and macrophage proteostasis, in a subject by administering a CMA activator to the subject. Enhancing proteostasis is a method of preventing or slowing advancement of an age-related neurodegenerative disease, atherosclerosis, or an inflammatory disease in a subject. The age related neurodegenerative disease can be Alzheimer A disease (AD), Lewy body dementia, Parkinson's disease (PD), Huntington's disease, Amyotrophic lateral sclerosis (ALS), Frontotemporal dementia (FTD). Spinocerebellar ataxias (SC As), or Progressive subcortical gliosis. The disclosure further provides a method of reducing the progression of beta-amyloid and/or tau pathology in the subject, and/or reduces pre-existing beta-amyloid and/or tau pathology in the subject.

### CROSS REFERENCE TO RELATED APPLICATIONS

This application claims priority of U.S. Provisional Appl. No. 63/177,504 filed Apr. 21, 2021, and U.S. Provisional Appl. No. 63/323,942 filed Apr. Mar. 25, 2022.

### BACKGROUND

Loss of proteostasis underlies the basis of multiple age-related degenerative disorders. Chaperone-mediated autophagy (CMA) activity, essential in the cellular defense against proteotoxicity, declines with age.

Maintenance of proteostasis is essential for normal cellular function and for adaptation to the always changing extracellular environment. Chaperones and the proteolytic systems are the major components of the proteostasis network. Gradual loss in functionality of some of these proteostasis pathways with age has been proposed to accelerate the course of degenerative conditions that afflict the elderly.

All cells rely on intracellular surveillance systems to maintain their proteome's homeostasis (proteostasis). These systems are especially important in neurons that, due to their postmitotic status, are highly sensitive to proteotoxic insult. Decreased neuronal protein quality control with age increases the risk of neurodegenerative diseases. In fact, presence of protein aggregates is a common feature in neurodegenerative patient brains. Interestingly, most elderly brains also display protein aggregation, even in absence of disease.

Defective autophagy, one of the components of the proteostasis network, associates with neurodegenerative diseases, including Parkinson's disease (PD) and Alzheimer's disease (AD). Macroautophagy has been proven necessary for maintenance of neuronal proteostasis and for protection against neurodegeneration.

Maintaining proteostasis is also important in vascular cells, vascular smooth muscle cells (VSMC), and macrophages. Cardiovascular disease (CVD) is the leading underlying cause of death worldwide accounting for more than 31.5% of total deaths. The main risk factors for the development of atherosclerosis—the most common cause of CV clinical events—such as obesity, hypertension, diabetes, and aging are rising in epidemic proportions due to changes in lifestyle and the growing elderly population. In atherosclerosis, hypercholesterolemia leads to vascular endothelial dysfunction and extravasation of atherogenic lipoproteins, resulting in increased adhesion and extravasation of monocytes from the circulation to the intima which progresses to atherosclerosis.

There exists a need for a method of maintaining proteostasis in neuronal and vascular cells, including in the neuronal cells of a patient at risk for a neurodegenerative disorder and vascular and macrophage cells in a patient at risk for cardiovascular disease or having atherosclerosis. This disclosure fulfills this need and provides additional advantages.

### SUMMARY

The disclosure provides a method of preventing or slowing advancement of an age related neurodegenerative disease in a subject in need thereof, comprising identifying an early symptom or biomarker of the neurodegenerative disease in the subject, and administering a therapeutically effective amount of a CMA activator to the subject, wherein the subject is asymptomatic or is in an early symptomatic stage of the age-related neurodegenerative disease.

The disclosure also provides a method of enhancing neuronal proteostasis in a subject in need of treatment for an age-related neurodegenerative disorder, comprising administering a CMA activator to the subject, wherein administering the CMA activator enhances neuronal proteostasis in the subject.

The disclosure provides a method of increasing Lamp 2A levels in neurons of a subject in need of treatment for an age-related neurodegenerative disorder, comprising administering a CMA activator to the subject, wherein administering the CMA activator increases Lamp 2A levels in the neurons of the subject.

This disclosure provides a method of delaying the onset of a neurodegenerative disorder in a patient comprising: determining the patient has a risk factor associated with developing the neurodegenerative disorder;

The disclosure provides a method of administering an amount of a Chaperone Mediated Autophagy (CMA) activator to the patient sufficient to increase CMA activity in the excitatory and/or inhibitory neurons of the patient; and thereby delaying the onset of the neurodegenerative disorder.

The disclosure provides a method of maintaining glycolytic activity in neurons of a patient. comprising administering an amount of a Chaperone Mediated Autophagy (CMA) activator to the patient sufficient to activate CMA in the excitatory and/or inhibitory neurons of the patient; and thereby maintaining glycolytic activity in the patient's neurons.

The disclosure provides a method of reducing the level of a marker Alzheimer's Dementia (AD) pathology or slowing the increase of a marker of AD pathology in a patient diagnosed as at risk of developing AD or in a patient diagnosed as having AD, comprising

Determining a first level of the marker of AD pathology in the patient; Administering an amount of a Chaperone Mediated Autophagy (CMA) activator to the patient sufficient to activate CMA in the neurons of the patient daily for a period of at least 3 months, at least 6 months, or at least 12 months; and Determining a second level of the marker of AD pathology in the patient after the administration of the CMA activator;

Comparing the first level and the second level of the marker AD pathology; and determining whether the level of the marker of AD pathology has decreased or slowed in the patient.

This disclosure provided a method of reducing gliosis or inflammation in the brain of a patient, comprising administering an amount of a Chaperone Mediated Autophagy (CMA) activator to the patient sufficient to activate CMA in the brain of the patient; and thereby reduce gliosis or inflammation brain of the patient.

This disclosure also provides a method of increasing proteostasis and/or gliosis in neurons of a patient having a neurodegenerative disorder, comprising administering an amount of a Chaperone Mediated Autophagy (CMA) activator to the patient sufficient to activate CMA in the neurons of the patient; and thereby increasing proteostasis and/or gliosis in the neurons of the patient.

The disclosure further provides a method of preventing protein aggregation in the neurons of a patient comprising Administering an amount of a Chaperone Mediated Autophagy (CMA) activator to the patient sufficient to decrease soluble protein accumulation in the neurons of the patient.

### BRIEF DESCRIPTION OF THE DRAWINGS

FIG. 1. Mice with CMA blockage display behavioral impairments. (A) Schematic of the generation of mice with systemic (whole body; L2A^{-/-}) or neuronal-specific (CaMKinase-II a Cre; CKL2A^{-/-}) deletion of LAMP2A (L2A). (B) Clasping, age-dependent change in clasping score [genotype effect: F_{2,63}=18.24, p<0.0001]. (C) Clasping, mean linear regression coefficient (±95% C.I.) on clasping scores. (D) Negative geotaxis test in CTR, L2A^{-/-} and CKL2A^{-/-} mice at 6 months. Quantification of latency. [Kruskal-Wallis statistic=9.987, p=0.0018]. (E) Short-term memory Y test in 6 months old CTR, L2A^{-/-} and CKL2A^{-/-} mice, Quantification of the fraction of time spent in the novel arm [F_{2, 12}=4.775, p=0.0298]. (E) Spontaneous locomotion in an open field in CTR, L2A^{-/-} and CKL2A^{-/-} mice at 6 months, quantification of the distance walked over 10 minutes. [Kruskal-Wallis statistic=9.006, p=0.0046]. (G) Stride length in CTR, L2A^{-/-} and CKL2A^{-/-} mice at 6 months, quantification. [Genotype effect: F_{2,79}=35.84, p<0.0001]. (H) Spatial working memory measured at 6 months by the percentage of spontaneous alternation in the Y maze for WT, L2A^{-/-}, and CKL2A^{-/-} mice. (I) Nest building test in 6 months old WT, L2A^{-/-}, and CKL2A^{-/-} mice, quantification is shown. [Genotype effect, Kruskal-Wallis statistic=8.814; p=0.0035]

All data are mean+s.e.m. and individual values are shown in D, E, F, G, H, and IP, . n=4-8 per timepoint/genotype. Comparisons were made using Kruskal-Wallis test followed by Dunn's post hoc test (D, F, H, I), one-way ANOVA followed by Tukey's post hoc test (E, P) or two way ANOVA followed by Sidak's post hoc test (H). *p<0.05, **p<0.005, ***p<0.0005.

FIG. 2. CMA deficiency in excitatory neurons leads to proteostasis collapse. (A) Lipofuscin autofluorescence in the hippocampus of CTR and CKL2A^{-/-} mice. Quantification of puncta per cell, (B) Immunostaining for K63-linkage ubiquitin in CTR and CKL2A^{-/-} mice hippocampal neurons, staining intensity distribution per cell body. Insert highlights the CA3 region. (C) Immunoblot for carbonyl groups to detect oxidized proteinsin the hippocampus of CTR and CKL2A^{-/-} mice. Representative immunoblots (top) and normalized densitometric quantification (bottom). (D) Comparative quantitative proteomics of CTR and CKL2A^{-/-} mice cortex, prone-to-aggregate proteins in the insoluble fraction (solid and discontinuous lines represent presence and absence of KFERQ-like motifs, respectively). Scale bars: 50 μm (B), 20 μm (A). Data are mean+s.e.m. and individual values (A; C). Comparisons were made with unpaired t-test (A-C) *p<0.05, **p<0.005, ***p<0.0005. See also FIG. 4.

FIG. 3. Consequence of systemic CMA blockage in brain proteostasis. (A) Lipofuscin autofluorescence and (B, C) immunostaining for K63-linkage ubiquitin in hippocampal neurons of 6 months old CTR and L2A^{-/-} mice. Quantification of number of fluorescent puncta per cell [t₈=4.012, p=0.0039] (A) or staining intensity distribution per cell body [t₁₉₈=9.945, p=p<0.0001] (B). Scale bar: 20 μm (A) 50 μm (B). n=20 cells per mice, 5 mice per genotype. (C) Higher magnification and XZ/YZ projections of immunostaining for K63-linkage ubiquitin to illustrate presence of protein aggregates (white arrowhead) in the CA3 region (left) and cellular area occupied by ubiquitin-positive aggregates (right). Overall cumulative distribution [Mann Whitney U=1209, p<0.0001] and mean per animal is shown as inset [t₈=4.398, p=0.023]. Scale bar: 10 μm.

FIG. 4. Neuronal CMA blockage did not induce gliosis or changes in macroautophagy. Samples from cortex and hippocampus of CKATG7^{-/-} mice are shown as positive control of (A) disrupted macroautophagy. Representative electron microscopy images of cortex (left, full field; right, examples of autophagosomes (APG) and autolysosomes (AUT)). (B) Bottom shows morphometric quantification of number of vesicles per field (left) and percentage of autophagic vacuoles (AV) at the APG and AUT state (right). (C)Low (left) and high magnification (right) images of CKL2A^{-/-} mice cortex to highlight accumulation of electrodense material (lighter arrows) and presence of autophagic vacuoles (darker arrows) in the proximity. Bottom shows examples of cytosolic and membrane surrounded (inside vesicles) electrodense material. Scale bar: 1 μm for full fields and 0.20 μm for insets. Data are mean±s.e.m. and individual values from n=4-5 per group. Comparisons were made using unpaired t-test.

FIG. 5. Blockage of CMA and macroautophagy in excitatory neurons leads to collapse of different subsets of the neuronal proteome. (A) Diagram of the two autophagic pathways blocked in excitatory neurons this study: macroautophagy and chaperone-mediated autophagy (right).

(B-C) Comparative quantitative proteomics of the insoluble fractions of CKL2A^{-/-} and CKATG7^{-/-} mice brains. (B)Venn diagram of proteins in the insoluble fractions, and (C) network visualization of gene ontology enrichment of insoluble proteins (showing similarity between nodes in CKATG7^{-/-} (protein catabolic processes and cell cycle) and other nodes shows similarity between nodes in CKL2A^{-/-} mice).

(D) Extracellular acidification rates (ECAR) in primary cortical neurons from CTR and CKL2A^{-/-} mice upon addition of (D) glycolytic properties calculated from the areas under the curve in ECAR. (E-F) Clathrin-mediated endocytosis related proteins in cortex of CTR and L2A^{-/-} mice at 6 months. (E,F) Quantifications of representative immunobots. (G) Transferrin uptake at 10 min in differentiated neuroblastoma cell lines transduced with empty vector (Control) or shL2A construct (L2A(−)). Representative images of Transferrin (light gray) and Hoechst dub (darker gray) (left). Inset: higher magnification and quantification of transferrin uptake expressed as folds of control (Ctr) cells. n=15-25 cells per condition (right). Scale bar 20 μm. (H) Arpc2 in cortex of CTR and L2A^{-/-} mice at 6 months. (H) Quantification of representative immunoblots. (I) Immunostaining for actin in hippocampal neurons of CTR and L2A^{-/-} mice at 6 months. (I) quantification of representative images. Scale bars: 20 μm (G, I). Data are mean±s.e.m and individual values are shown in D, E, F, H, and I. Comparisons were made using unpaired t-test (D, E-I) *p<0.05, **p<0.005, ***p<0.0005.

FIG. 6. CMA activity is inhibited in a tauopathy mouse model and Alzheimer's disease patients' brains. (A-B) CMA (measured as the number of fluorescent puncta per cell) in CA1 pyramidal neurons (A) and GFAP-positive astrocytes (B) in the hippocampus of CTR and hTauP301L expressing mice (Tau) at 12 months. (A,B) Distribution of the number of KFERQ-Dendra⁺ puncta per cell in CTR and Tau mice (left) and mean number of puncta per cell per animal (right) (right). Dendra values are from 9-17 (A) or 10-19 (B) individual cell from 3-5 animals per genotype. Scale bar: 50 μm. (C) Normalized expression (z scoring within each cell type) of CMA network components (organized in functional groups and colored dots indicate the effect of a given element on CMA activity; Green: positive element; Red: negative element). (D) CMA activation score of excitatory (Excit.) and inhibitory (Inhib.) neurons (D) and of astrocytes (Astro.), microglia (Microg.) and oligodendrocytes (Oligo.) (E, F) Negative correlation between CMA activation score in excitatory neurons and pathology markers using Braak pathology staging (E), and NIA-Reagan score (F). Data are mean±s.e.m. in A, B, and D and individual values in A, B, E, F. Comparisons were made using Kruskal-Wallis test followed by Dunn's post hoc test (A, B), two-way ANOVA followed by Sidak's multiple comparisons test (D) or Pearson correlation test (I, J).**p<0.005, ***p<0.0005.

FIG. 7. Mouse model of CMA deficiency AD-related proteotoxicity with CMA. (A) Immunoblot for the indicated proteins in brains from control, L2A^{-/-}, 3× Tg and 3× Tg-L2A^{-/-} mice at 12 months of age. Ponceau staining of the membrane is shown as protein loading control. (B) Immunoblot for K63-linkage of ubiquitinated proteins (left) and densitometric quantification (right) in brains of CTR, L2A^{-/-}, Tg and Tg-L2A^{-/-} mice at 12 months [Genotype effect: F_{3,32}=5.615, p=0.0033]. n=9 mice per genotype. (C-E) Volcano plot of the quantitative proteomic analysis of brain from L2A^{-/-} (C), Tg (D) and Tg-L2A^{-/-} (E) compared with CTR mice brains. The number in the top left corner indicates the number of significant hits. Red dots: differentially expressed proteins (adj. p<0.05).

FIG. 8. Loss of CMA in neurons accelerates pathology in a mouse model of Alzheimer's disease-related proteotoxicity. (A) Immunostaining for Aβ (green) and S422-phosphorylated (pS422) tau (red) and Hoechst staining (blue) in the hippocampus of Tg and Tg-L2A^{-/-} mice. Montages of individual images from the scanning of whole brain slices are shown. Right shows higher magnification images of the dorsal hippocampus. Insets show boxed areas at higher magnification. Scale bar: 1500 μm. (B-H) Immunoblots for the indicated proteins in brains of 12 months old CTR, L2A^{-/-}, Tg and Tg-L2A^{-/-} mice (B) and densitometric quantifications expressed as folds of CTR (C,F) or folds of Tg (D, E, G, H) for: endogenous murine tau (C), human tau (D), S202/T205-phosphorylated tau (AT8)(E), APP (F), C-terminal fragments (CTFs) of APP (G) and αCTFs (H). n=9 mice per genotype. (I) ELISA for Aβ42 of low-speed supernatants of hippocampus from Tg and Tg-L2A^{-/-} mice. n=8-10 mice per genotype. (J-K) AlphaLISA of low-speed supernatants of hippocampus from Tg and Tg-L2A^{-/-} mice for S422-phosphorylated (pS422) tau (J) and aggregated tay (K). Left panels show time course and right panels show regression coefficient (+95% C.I.). n=3-10 mice per genotype/timepoint. Data are mean±s.e.m. (linear regression panels) and individual values (all other panels). Comparisons were made using Kruskal-Wallis test followed by Dunn's post hoc test (C, F), unpaired t-test (D,E,G,H,I) or two-way ANOVA followed by Sidak's post-hoc analysis (J). * p<0.05, **p<0.005, ***p<0.0005.

FIG. 9. Neuronal loss of CMA has a synergistic deleterious impact on Alzheimer's disease-related brain proteotoxicity. (A) Heatmap and hierarchical clustering analysis based on changes in protein abundance between genotypes (B) Network visualization of gene ontology enrichment analysis of proteins specifically modified in Tg-L2A^{-/-} mice brains. (C) Rank-rank hypergeometric overlap plots to show similarity in enriched proteins between both asymptomatic (AsymAD) and symptomatic AD (AD) cases and L2A^{-/-}, Tg and Tg-L2A^{-/-} mice. (K) Human Aβ-correlated proteins (from Bai et al. 2020) validated in L2A^{-/-}, Tg and Tg-L2A^{-/-} mice. Numbers at the bottom indicated the number of consistent changes between AD and mouse proteome.

FIG. 10. Characterization of the brain permeable chemical activator of CMA and effect in a mouse model of Frontotemporal dementia-related proteotoxicity. (A) Chemical structure of AR7 and CA77.1. (B) Quantification of the effect of adding increasing concentrations of AR7 and CA77.1 to NIH3T3 cells stably expressing the KFERQ-PS-Dendra reporter. CMA activity was quantified after 16 h treatment as the average of fluorescent puncta per cell. (n>800 cells per condition). (C) Autophagic flux in NIH3T3 cells incubated at 37° C. for 16 h with 20 □M AR7 or CA77.1 or without additions (none). Where indicated lysosomal protease inhibitors (PI) were added to the cells 6 h before lysis. Left: representative immunoblot. Actin is shown as loading control. Right: quantification of LC3-II flux as the difference in LC3-II levels in cells treated with PI relative to untreated. Values are presented as folds those in untreated cells that were given an arbitrary value of 1. n=5 independent experiments. (D) Quantification of the effect of adding increasing concentrations of AR7 and CA77.1 to NIH3T3 cells stably expressing the KFERQ-PS-Dendra reporter. CMA activity was quantified after 12 h or 24 h treatment as the average of fluorescent puncta per cell. (n>800 cells per condition). (E) Pharmacokinetic analysis of CA77.1 after p.o. (oral, 10 mg/Kg bw) and i.v. (intravenous, 1 mg/kg bw) administration in mice. n=3 mice per time point. (F) Summary of pharmacokinetic parameters of CA77.1 in mice brain. (G) Mouse blood count at the end of 4 months daily oral administration of vehicle (Veh) or CA77.1 (CA). n=6 mice in each group. (H-J) Representative images of sections of H&E stained liver (B), lung (C) and kidney (D) sections from the same mice. Two magnifications are shown. In D, details of glomeruli and tubules are shown in the higher magnification images. (K) Distribution of four types of S202/T205-phosphorylated tau staining described in Shi et al. 2017. Numbers indicate the number of mice belonging to each group. n=10 mice per group. (L-N) Immunoblot for AT8 tau on the Sarkosyl-insoluble fraction of CTR and PS19 mice administered Veh or CA for 6 months. HMW: high-molecular weight species of AT8. Representative immunoblot of 4-5 mice per genotype and treatment (L) and densitometric quantification of AT8 as monomer [Kruskal-Wallis statistic=20.48, p<0.0001] (M) and as HMW-species [F_{2.26}=7.183, p=0.0033] (N). (O, P) Quantification of the number of Iba1-positive microglial cells per mm² in the hippocampus [Kruskal-Wallis statistic=10.55, p=0.0051] (O) and piriform cortex (P) of CTR and PS19 mice treated with Veh or CA for 6 months. Images shown in FIG. 14N. Mean+s.e.m and individual values. Comparisons were made using One-way ANOVA followed by Tukey post-hoc analysis *p<0.05.

FIG. 11. Chemical activation of CMA improves behavior and neuropathology in a mouse model of Frontotemporal dementia-related proteotoxicity. (A) Schematic of regimen of CMA activator (CA) administration to mice overexpressing human P301S tau (PS19). (B, C) Spontaneous locomotion in an open field of 9-month-old CTR or PS19 mice administered vehicle (Veh) or CA. Representative tracks (B) and total distance traveled in 10 min (C). (D-H) Immunostaining for MC1 tau in the hippocampus, the amygdala and the piriform cortex of 9 months old CTR, PS19 +/− CA. Representative images of the indicated brain regions (D). Scale bars: hippocampus: 500 μm, amygdala and piriform cortex: 200 μm. Quantification of the area stained in the hippocampus (E), (F-J) Immunoblots for tau and phosphorylated tau. Representative immunoblots and densitometric quantifications for the indicated proteins: S422-phosphorylated tau (G), S202/T205-phosphorylated tau (AT8) (H), MC1 tau (I) and total tau (J). (K-M) Immunostaining for Iba1 (K) and quantification of average cell size in the hippocampus (L) and the piriform cortex (M). Scale bars: hippocampus: 500 μm and piriform cortex: 200 μm. Data are mean+s.e.m. Individual values are shown for n=9-10 mice per genotype and treatment in C, E, G-M). Comparisons were made using one-way ANOVA followed by Tukey's post-hoc analysis (C, E, G, I-M) or Kruskal-Wallis test followed by Dunn's post hoc test (H). *p<0.05, **p<0.005, ***p<0.0005.

FIG. 12. Chemical activation of CMA improves behavior and neuropathology in a mouse model of Alzheimer's disease-related proteotoxicity. (A) Schematic of regimen of CMA activator (CA) administration to TauPS2APP (Tg) mice. (B-D) Performance of Tg mice administered vehicle (Veh) or CMA activator (CA) in the novel object recognition test (B, left plot: % of time dedicated to the novel object; right plot: discrimination index), the elevated plus maze (C) and the forced swim test (D). (E) Clasping score in Tg mice +/− CA: time course of clasping score increase (left) and mean linear regression coefficient (right). (F) Performance of Tg mice +/− CA in horizontal grid test. (G-O) Immunostaining of immature amyloid plaques (MOAB2), mature amyloid depositions (6E10), β-sheet marker (Thioflavin S) and Threonine231-phosphorylated tau (pThr231 tau) in the dorsal hippocampus of Tg mice +/− CA. Montages of individual images from the scanning of whole brain slices are shown. Inset: Higher magnification of pThr231 tau staining. Scale bar: 200 μm. Quantification of images of montages of individual images from the scanning of whole brain slices of percentage of area positive and plaque size for MOAB2 (G, J), 6E10 (H, K) or Thioflavin S (I, L). Relationship between number of plaques and average plaque size for different markers of plaque maturity (ThS>6E10>MOAB2) (N). Quantification of percentage of area positive for pThr231 tau (N) and of the overlap between pThr231 tau staining and 6E10 staining (O). All quantifications (except clasping—E) were done at 12 months, in 4-6 mice per group. Data are mean±s.e.m. Comparisons were made using unpaired t-test (B-D, F, G-O), Two-way ANOVA test followed by Sidak's post-hoc analysis (E). *p<0.05, **p<0.005, ***p<0.0005.

FIG. 13. Effect of chemical activation of CMA in a mouse model of Alzheimer's disease-related proteotoxicity. (A-D) Quantification of size of plaques positive for MOAB2 (t₇=3.181, p=0.0155) (A), 6E10 (B) or Thioflavin S (ThS) (C) and of T231-phosphorylated tau pathology (t₈=2.382, p=0.0444) (D) in brain cortex from Tg mice administered Veh or CA for 4 months. (E) Summary heatmap highlighting the extent of pathology reduction upon CMA activation in cortex and dorsal hippocampus. * highlights significant difference between Veh and CA administered Tg mice. (F) Immunoblot for S202/T205-phosphorylated (AT8) tau in the brain of Tg mice treated with Veh or CA. Representative immunoblot (left) and densitometric quantification (t₈=2.425, p=0.0415) (right). Values were normalized to ponceau and are expressed as folds of Veh treated mice. (G) Number of GFAP-positive astrocytes per area unit (t₈=4.058, p=0.0036). (H-I) Representative images of immunostaining of inflammation (microglial marker: Iba1—Green), amyloid pathology (MOAB2—Magenta) and nuclear staining with DAPI (blue) in the dorsal hippocampus of Tg mice administered Veh or CA. Montages of individual images of the dorsal hippocampus from the scanning of whole brain slices are shown. Quantification of number of Iba1-positive microglial cells per area unit (t₈=2.041, p=0.0755) (left) and mean cell size (t₈=2.079, p=0.0713) (right) (H) and colocalization coefficient between microglia (Iba1) and amyloid plaques (MOAB2) (I) (Mann Whitney U=6, p=0.2571). Mean±s.e.m. and individual values are shown. Comparisons were made using unpaired t-test analysis. *: p<0.05, **: p<0.005.

FIG. 14. CMA deficiency aggravates atherosclerosis in a murine experimental model. (A) CMA activity in aorta from KFERQ-Dendra2 mice subjected to a pro-atherosclerotic treatment (injected with AAV8 PCSK9 and maintained for 12 weeks on Western-type diet, WD. Representative images of aorta sections co-stained with LAMP1 to highlight endolysosomal compartments. Insets: boxed areas at higher magnification. Arrows: fluorescent puncta. Individual and merged channels of the boxed region at higher magnification are shown. Arrows: Dextran+Dendra+puncta and Dextran+only puncta. (B) Levels of LAMP-2A at the indicated times of the pro-atherosclerotic intervention. (C,D) Circulating lipids in wild type (WT) and LAMP-2A null mice (L2AKO) subjected to the pro-atherosclerotic challenge for 12 weeks. Circulating total cholesterol (C) and triglycerides (TG) (D). (E) Quantification of plaque area in representative images of aortas, plaque stage index (F), and sirius red positive area (G). (H) Quantification of representative images of aortas immunostained for CD68 macrophages. Experiments in panel A, were repeated 3 times with similar results. All data were tested for normal distribution using D'Agostino and Pearson normality test. Variables that did not pass normality test were subsequently analyzed using Mann-Whitney rank-sum test. All other variables were tested with the Student's t-test. *p<0.05, **p<0.01, ***p <0.005 and ****p<0.0001.

FIG. 15. CMA blockage makes VSMC vulnerable to lipotoxicity and promotes their dedifferentiation. (A) Intracellular levels of diLDL-derived fluorescence in VSMC from wild type (WT) and LAMP-2A null mice (L2AKO). (n=3, >45 cells per experiment in 3 different experiments). (B) Cytotoxicity in the same cells in response to increasing concentrations of LDL (two-way ANOVA after Bonferroni's post hoc test, F=2.862; P=0.9872 for interaction; F=2.205; P=0.1570 for LDL concentration, F=21.93; P=0.0002 for genotype, n=5)). (C) Changes in mRNA levels of different markers of cell identity, macrophage-related and cholesterol pathway in the same VSMC stimulated with LDL or maintained in a LPDS (CTRL) (pool of 3 individual experiments).

FIG. 16. CMA blockage leads to exacerbated pro-inflammatory phenotype in macrophages. (A) Levels of iNOS and COX-2 proteins in BMDM from wild type (WT) and LAMP-2A null mice (L2AKO) cultured without additions (control, CTRL) or stimulated with IFNγ/LPS. Representative immunoblot (A) and densitometric quantification of representative immunoblot expressed as folds over WT levels (n=5 for iNOS and 3 for COX2). Ponceau red is shown as loading control. (B) mRNA levels of iNOS and Cox2 in the same cells expressed as folds over untreated (CTR).(n=4). (C) Number of pro-inflammatory subtype of monocytes (L) in WT and L2AKO mice (n=14 WT and n=16 L2AKO). All data, when applicable, were tested for normal distribution using D'Agostino and Pearson normality test. Variables that did not pass normality test were subsequently analyzed using Mann-Whitney rank sum test. All other variables were tested with the Student's t-test. All values are mean±SEM. Individual values are shown also in i−1. *p<0.05, **p<0.01, ***p<0.005 and ****p<0.001.

FIG. 17. Characterization of CMA-deficient macrophages. (A) Heat map of changes in levels of the proteome of lysosomes from WT for mouse exposed to IFNγ/LPS (F) upon inhibition of lysosomal proteolysis. (B,C) Predicted activation in BMDM L2AKO cells of the LPS pathway due to accumulation of CMA substrates (B) and the inflammatory response in IFNγ/LPS treated cells (C) using the IPA software. All data, when applicable, were tested for normal distribution using D'Agostino and Pearson normality test. Variables that did not pass normality test were subsequently analyzed using Mann-Whitney rank-sum test. All other variables were tested with the Student's t-test. Graphs represent mean+SEM. *p<0.05.

FIG. 18. CMA changes in aorta of atherosclerotic patients with disease. Average and individual values in all samples independent of gender (A) and representative immunoblot (top) and values in females only (B). Ponceau red is shown as loading control. (C) CMA activation score (C) calculated from normalized expression data between stable and unstable atherosclerotic plaques. Decrease of the score indicates a predicted transcriptional inhibition of the pathway [t41=1.612, p=0.1146]. Normalized expression of individual component of the CMA network in RNAseq from stable and unstable atherosclerotic plaques. All data, when applicable, were tested for normal distribution using D'Agostino and Pearson normality test. Variables that did not pass normality test were subsequently analyzed using Mann-Whitney rank sum test. All other variables were tested with the Student's t-test. Individual patient values and mean±SEM are shown. *p<0.05 and **p<0.01. n=36 for the first study. n=15-20 for the second study.

FIG. 19. Genetic upregulation of CMA ameliorates disease in an atherosclerosis murine experimental model. (A, B) Circulating lipids in control mice (CTRL) and in mice systemically expressing a copy of human LAMP-2A (hL2AOE) subjected to a pro-atherosclerotic intervention (injected with AAV8 PCSK9 and maintained for 12 weeks on the Western-type diet). Cholesterol profile (A) and Triglyceride profile (B) are shown. n=9 CTRL, n=8 hL2AOE. (C-E) Properties of the plaques from aortas of the same mouse groups. Quantification of plaque area from representative images of aortas stained for hematoxylin and eosin (H&E) (C), size of the necrotic core (D), and calcification presence (E). n=9 CTRL, n=8 hL2AOE. (F,G) Principal component analysis of 12 variables measured in CTRL and hL2AOE mice. Each dot represents a single animal (F). Ellipses are the 95% confidence interval around the center of mass of a given experimental group. Bar plot represents mean+/−s.e.m. of PC1 score for each experimentalgroup (G). n=9 CTRL, n=8 hL2AOE. *: Student t-test between CTRL and hL2AOE t15=2.152, p=0.048. Individual values and mean+SEM are presented in all quantifications. All data were tested for normal distribution using D'Agostino and Pearson normality test. Variables that did not pass normality test were subsequently analyzed using Mann-Whitney rank-sum test. All other variables were tested with the Student's t-test. **p<0.01.

### DETAILED DESCRIPTION

Prior to setting forth the invention in detail, it may be helpful to provide definitions of certain terms to be used in this disclosure. Compounds are described using standard nomenclature. Unless defined otherwise, all technical and scientific terms used herein have the same meaning as is commonly understood by one of skill in the art to which this invention belongs. Unless clearly contraindicated by the context each compound name includes the free acid or free base form of the compound as well as all pharmaceutically acceptable salts of the compound.

The terms “a” and “an” do not denote a limitation of quantity, but rather denote the presence of at least one of the referenced item. The term “or” means “and/or”. The open-ended transitional phrase “comprising” encompasses the intermediate transitional phrase “consisting essentially of” and the close-ended phrase “consisting of.” Claims reciting one of these three transitional phrases, or with an alternate transitional phrase such as “containing” or “including” can be written with any other transitional phrase unless clearly precluded by the context or art. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in any suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

“Pharmaceutical compositions” are compositions comprising at least one active agent, such as a compound or salt of a CMA Activator, and at least one other substance, such as a carrier. Pharmaceutical compositions optionally contain one or more additional active agents. When specified, pharmaceutical compositions meet the U.S. FDA's GMP (good manufacturing practice) standards for human or non-human drugs.

Currently, diagnosis of neurodegenerative disease such as Alzheimer's disease (AD)relies on identifying mental decline, at which point significant brain damage has been done. Similarly, Parkinson's disease (PD) is identified by symptoms such as shaking or tremors, slowness of movement (bradykinesia), stiffness or rigidity of the arms and legs, and/or balance issues (postural instability). PD is a progressive disease in which the symptoms worsen over time. The methods described herein provide for preventing or slowing advancement of an age-related neurodegenerative disease in a subject in need thereof when the subject is asymptomatic or is in an early symptomatic stage of the age-related neurodegenerative disease. Early intervention may help to prevent the progression of symptoms and delay progression to late-stage age-related neurodegenerative disease.

In an aspect, a method of preventing or slowing advancement of an age-related neurodegenerative disease in a subject in need thereof comprises identifying an early symptom or biomarker of the neurodegenerative disease in the subject, and administering a therapeutically effective amount of a CMA activator to the subject. In an aspect, the subject is asymptomatic or is in an early symptomatic stage of the age-related neurodegenerative disease.

Administering the CMA activator can reduce the progression of beta-amyloid and/or tau pathology in the subject, and/or reduce pre-existing beta-amyloid and/or tau pathology in the subject. Prior to the experiments described herein, it was not expected that CMA modulation would affect beta-amyloid and/or tau pathology. The method optionally further comprises determining the progression of beta-amyloid and/or tau pathology by positron emission tomography (PET) and/or magnetic resonance (MR) imaging. ¹¹C-labeled Pittsburgh Compound-B ([¹¹C]PiB), also known as 2-(4-N-[¹¹C]methylaminophenyl)-6-hydroxybenzothiazole, [¹⁸F]Florbetapir ([¹⁸F]FBP), which is also known as ¹⁸F-AV-45 or 4-{(E)-2-[6-(2-{2-[2-(18F)Fluoroethoxy]ethoxy}ethoxy)-3-pyridinyl]vinyl}-N-methylaniline, [¹⁸F]Florbetaben ([¹⁸F]FBB), and [¹⁸F]Flutemetamol ([¹⁸F]FMT) are radiotracers for beta-amyloid {ET imaging. The PET ligand [¹⁸F]AV-1451 binds tau-positive inclusions. The levels of tau protein (total tau or phosphorylated tau) or beta-amyloid (e.g., Aβ42) in the plasma or cerebrospinal fluid (CSF) of the subject can also be used to determine the progression of beta-amyloid and/or tau pathology.

It was also unexpectedly found that CMA mdosulators have an effect on gliosis, defined herein as progression of glial cells. In an aspect, administering the CMA inhibitor reduces gliosis in the brain of the subject, for example as determined by positron emission tomography (PET) and/or magnetic resonance (MR) imaging.

Progressive subcortical gliosis is a chromosome-17-linked dementia with unique pathologic features including fibrillary astrocytosis. Early symptoms include personality and emotional changes, lack of judgment and insight, deterioration in social behavior, delusions, paranoia, auditory and visual hallucinations, and depression.

In an aspect, the method further comprises detecting an increase in neuronal glycolysis after administering the CMA activator. Unexpectedly, CMA activation has been shown to increase glycolysis.

In another aspect, a method of enhancing neuronal proteostasis in a subject in need of treatment for an age-related neurodegenerative disorder comprises administering a CMA activator to the subject, wherein administering the CMA activator enhances neuronal proteostasis in the subject. In an aspect, administering the CMA activator reduces the progression of beta-amyloid and/or tau pathology in the subject, and the method optionally comprises determining the progression of beta-amyloid and/or tau pathology by positron emission tomography (PET) and/or magnetic resonance (MR) imaging, or by tau protein (total tau or phosphorylated tau) or beta-amyloid (e.g., Aβ42) in the plasma or cerebrospinal fluid (CSF) of the subject. In an aspect, the method further comprises detecting an increase in neuronal glycolysis after administering the CMA activator.

In yet another aspect, a method of increasing Lamp 2A levels in neurons of a subject in need of treatment for an age-related neurodegenerative disorder, comprises administering a CMA activator to the subject, wherein administering the CMA activator increases Lamp 2A levels in the neurons of the subject.

In another aspect the disclosure provides a method of protecting a subject against developing atherosclerosis, significantly reducing the likelihood of a subject at risk for atherosclerosis from developing atherosclerosis, slowing the progression of atherosclerosis in a subject having atherosclerosis, or decreasing atherosclerosis in a subject having atherosclerosis by administering a therapeutically effective amount of a CMA activator to the subject. The disclosure also provides a method of protecting vascular cells, including smooth muscle vascular cells in a subject by administering a CMA activator to the subject. The disclosure also provides a method of preventing or decreasing macrophages having a pro-inflammatory phenotype, for example in a subject having atherosclerosis, include asymptomatic atherosclerosis, comprising administering a CMA activator to the subject.

Macrophages in atherosclerotic lesions actively participate in lipoprotein ingestion and accumulation giving rise to foam cells filled with lipid droplets. Accumulation of foam cells contributes to lipid storage and atherosclerotic plaque growth and chronic inflammatory conditions.

Pathologic inflammatory conditions are frequently correlated with dynamic alterations in macrophage activation, with classically activated M1 cells associated with promoting and sustaining inflammation and M2 cells implicated in resolution or smoldering chronic inflammation (DOI: 10.2174/1874467215666220324114624). Reduced CMA in macrophages promotes a bias in macrophage differentiation toward M1 (pro-inflammatory phenotype).

### Pharmaceutical Preparations

Compounds disclosed herein can be administered as the neat chemical, but are preferably administered as a pharmaceutical composition. Accordingly, the disclosure provides pharmaceutical compositions comprising a compound or pharmaceutically acceptable salt of a CMA modulator, such as a CMA Activator, together with at least one pharmaceutically acceptable carrier. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of a CMA Activator and optionally from about 0.1 mg to about 2000 mg, from about 10 mg to about 1000 mg, from about 100 mg to about 800 mg, or from about 200 mg to about 600 mg of an additional active agent in a unit dosage form.

Compounds disclosed herein may be administered orally, topically, parenterally, by inhalation or spray, sublingually, transdermally, via buccal administration, rectally, as an ophthalmic solution, through intravitreal injection or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution for topical or intravitreal injection. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert, or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present disclosure.

The pharmaceutical compositions/combinations can be formulated for oral administration. These compositions contain between 0.1 and 99 weight % (wt. %) of a CMA Activator and usually at least about 5 wt. % of a compound of a CMA Activator. Some embodiments contain from about 25 wt. % to about 50 wt. % or from about 5 wt. % to about 75 wt. % of the compound of a CMA Activator.

### Methods of Treatment

The disclosure also provides methods of selectively activating chaperone-mediated autophagy (CMA) in a subject in need thereof comprising administering to the subject a CMA Activator in an amount effective to activate CMA in the subject.

The subject can have, for example, a neurodegenerative disease, such as tauopathies, (Frontotemporal Dementia, Alzheimer's disease), Parkinson's Disease, Huntington's Disease, prion diseases, amyotrophic lateral sclerosis, retinal degeneration (dry or wet macular degeneration, retinitis pigmentosa, diabetic retinopathy, glaucoma, Leber congenital amaurosis), diabetes, acute liver failure, non-alcoholic steatohepatitis (NASH), hepatosteatosis, alcoholic fatty liver, renal failure and chronic kidney disease, emphysema, sporadic inclusion body myositis, spinal cord injury, traumatic brain injury, fibrosis (liver, kidney, or lung), a lysosomal storage disorder, a cardiovascular disease, and immunosenescence. Lysosomal storage disorders include, but are not limited to, cystinosis, galactosialidosis, and mucolipidosis. The subject may also have a disease or condition in which CMA is upregulated such as cancer or Lupus. The subject can have reduced CMA compared to a normal subject prior to administering the compound. Preferably, the compound does not affect macroautophagy or other autophagic pathways. In macroautophagy, proteins and organelles are sequestered in double-membrane vesicles and delivered to lysosomes for degradation. In CMA, protein substrates are selectively identified and targeted to the lysosome via interactions with a cytosolic chaperone and cross the lysosomal membrane through a translocation complex.

The disclosure also provides a method of protecting cells from oxidative stress, hypoxia, proteotoxicity, genotoxic insults or damage and/or lipotoxicity in a subject in need thereof comprising administering to the subject any of the compounds disclosed herein, or a combination of a CMA Activator, in an amount effective to protect cells from oxidative stress, hypoxia proteotoxicity, genotoxic insults or damage, and/or lipotoxicity. The subject can have, for example, one or more of the chronic conditions that have been associated with increased oxidative stress and oxidation and a background of propensity to proteotoxicity. The cells being protected can comprise, for example, cardiac cells, kidney and liver cells, neurons and glia, myocytes, fibroblasts and/or immune cells. The compound can, for example, selectively activate chaperone-mediated autophagy (CMA). In one embodiment, the compound does not affect macroautophagy.

In a specific aspect, the subject is suffering from mild cognitive impairment. As used herein, mild cognitive impairment is the stage between the expected cognitive decline due to aging and the more serious decline of dementia. Forgetfulness, losing train of thought or difficulty following conversations, difficulty making decisions, getting lost in familiar environments and poor judgment can be signs of mild cognitive impairment. Mild cognitive impairment can progress to Alzheimer's disease or other forms of dementia.

Exemplary age-related neurodegenerative diseases include Alzheimer's disease (AD), Lewy body dementia, Parkinson's disease (PD), Huntington's disease, Amyotrophic lateral sclerosis (ALS), Frontotemporal dementia (FTD), Spinocerebellar ataxias (SCAs), Progressive subcortical gliosis, and the like.

When the age-related neurodegenerative disease is AD, the subject for the methods described herein subject may not suffer from dementia. Exemplary early symptoms of AD include memory loss and/or confusion, difficulty concentrating, difficulty completing daily tasks, time and/or place confusion, difficulty with visual images and/or spatial relationships, difficulty conversing, misplacing objects, poor judgment, withdrawal from activities, changes in mood and personality. Exemplary biomarkers for AD are tau protein (total tau or phosphorylated tau) or beta-amyloid (e.g., Aβ42) in the plasma or cerebrospinal fluid (CSF) of the subject.

In Lewy body dementia, protein deposits called Lewy bodies develop in nerve cells in the regions of the brain involved in cognition, memory and movement. Early symptoms of Lewy body dementia include loss of small, acting out while dreaming, visual hallucinations, confusion, difficulty maintaining attention, memory loss, changes in handwriting, muscle rigidity, falling, and drowsiness. Currently there are no verified biomarkers for Lewy body dementia.

PD is a progressive nervous system disorder that affects movement. Exemplary early symptoms of PD include slight tremors in the fingers, thumbs, hand or chin; small handwriting (also called micrographia); loss of smell; difficulty sleeping including sudden movements in sleep; difficulty moving or walking; constipation; a soft or low voice; facial masking; dizziness or fainting; and/or stooping, leaning or slouching while standing. Currently there are no verified clinical biomarkers for PD.

Huntington's disease is a genetic disorder that causes progressive degeneration of nerve cells in the brain. Early symptoms of Huntington's disease include difficulty concentrating, memory lapses, depression, clumsiness, small involuntary movements and mood swings. Mutant Huntington protein (mHtt) is a biomarker for Huntington's disease. Subjects who carry the Huntington mutation can be treated by the methods described herein.

ALS is a rare, progressive disease involving the nerve cells responsible for controlling voluntary movements. Early symptoms of ALS include muscle twitches in the arm, leg, shoulder or tongue; muscle cramps; stiff muscles; muscle weakness of the arm, leg, neck or diaphragm; slurred and nasal speech; and difficultly chewing or swallowing. Currently there are no validated biomarkers for ALS.

FTD, sometimes called Pick' disease, is a group of neurological disorders in which nerve cells in the front and temporal lobes of the brain are lost. Early symptoms of FTD include changes to personality and behavior and/or difficulties with language. Clinically, differentiating between FTD and AD is challenging.

Spinocerebellar ataxias (SCAs) are progressive disorders in which the cerebellum slowly degenerates, often accompanied by degenerative changes in the brainstem and other parts of the central nervous system. Early symptoms of SCAs are problems with coordination and balance, speech and swallowing difficulties, muscle stiffness, weakness of the muscles that control eye movement, and cognitive impairment. SCA1, SCA2, SCA3, SCA6, SCA7 and SCA17 share the same pathogenic mechanism of CAG trinucleotide repeat expansions encoding elongated polyglutamine tracts. There is no serum biomarker for SCAs.

In an embodiment the subject is a mammal. In certain embodiments the subject is a human, for example a human patient undergoing medical treatment. The subject may also be a companion a non-human mammal, such as a companion animal, e.g. cats and dogs, or a livestock animal.

For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids (e.g., blood, plasma, serum, cellular interstitial fluid, cerebrospinal fluid, saliva, feces and urine) and cell and tissue samples of the above subjects will be suitable for use.

An effective amount of a pharmaceutical composition may be an amount sufficient to inhibit the progression of a disease or disorder, cause a regression of a disease or disorder, reduce symptoms of a disease or disorder, or significantly alter a level of a marker of a disease or disorder.

An effective amount of a compound or pharmaceutical composition described herein will also provide a sufficient concentration of a CMA Activator when administered to a subject. A sufficient concentration is a concentration of the CMA Activator in the patient's body necessary to prevent or combat a CMA mediated disease or disorder or other disease or disorder for which a CMA Activator is effective. Such an amount may be ascertained experimentally, for example by assaying blood concentration of the compound, or theoretically, by calculating bioavailability.

Methods of treatment include providing certain dosage amounts of a CMA Activator to a subject or patient. Dosage levels of each compound of from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a CMA Activator are provided daily to a patient. Frequency of dosage may also vary depending on the compound used and the particular disease treated. However, for treatment of most diseases and disorders, a dosage regimen of 4 times daily or less can be used and in certain embodiments a dosage regimen of 1 or 2 times daily is used.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

In an embodiment, the invention provides a method of treating a lysosomal storage disorder in a patient identified as in need of such treatment, the method comprising providing to the patient an effective amount of a CMA Activator. The CMA Activator may be administered alone as the only active agent, or in combination with one or more other active agent.

### EXAMPLES

### General Methods

### Animal Models

Male wild-type mice (C57BL/6J) or transgenic for CaMKIIα-Cre (B6.Cg-Tg(Camk2α-cre)T29-1Stl/J, Jackson Laboratory), LAMP-2A^{flox/flox} (Schneider et al., 2014), Tau^{P301L} (line pR5), Tg (APP^{Swe}; PS2^{N141I}; Tau^{P301L}), PS19 (Tau^{P301S}) (Yoshiyama et al., 2007) and Atg7^{f/f} were used in the study. Conditional LAMP-2A deletion was generated by breeding LAMP-2A^{flox/flox} with the transgenic Cre mice of interest. Knockout for L2A in the whole body (L2A^{-/-}) was generated by insemination of a wild-type female with spermatozoids with L2A floxed to excise this gene in all tissues in the offspring. Male littermate wild type and only L2A^{f/f} were separately analyzed for each test and because no differences were detected among them, they were grouped in the results as “control” (CTR) for the experimental group (^{CamKII□} CreL2A^{f/f} or L2A^{-/-}). All mice were genotyped at weaning and genotyping was re-confirmed postmortem to correct for any possible misplacement during husbandry. Mice were all in the C57BL/6J background and maintained under specific pathogen-free conditions in ventilated cages with no more than 5 mice per cage. Only males were used in this study due to the complexity in generating the quadruple transgenic mouse model with L2A knock out in homozygosis (for which we took advantage of the location of the Lamp2 gene in the X chromosome). Age of the animals was 4-6 months in most experiments except when otherwise indicated in text, figures and figure legends. Animals were maintained at 19-23° C. in 12 h light/dark cycle. Mice were fed ad libitum. CA77.1 was administered as sucralose jelly pellets for a daily dose of 30 mg/Kg body weight whereas the vehicle treated group received the same sucralose jelly pellet without drug. Briefly, for preparation of the jelly pellets the final amount of the compound per day (adjusted per animal weight) was dissolved in ethanol and then mixed with a warm gelatin solution (100 mg/ml, 10 mg/ml sucralose in water), that was poured into 24 well flat bottom plates for solidification. To avoid animal stress and competition for the pellets, animals were separated with a grid spacer in the same cage that they were housed, eating of the pellet was monitored and the spacer was removed as soon as all mice consumed the pellet (average time 2 min). Sentinel animals were included in each study to determine brain exposure at the end of treatment. These animals received the same batch of jelly pellets in parallel to the experimental group. Animals were assigned randomly to the vehicle and placebo groups and no animals were eliminated from the study. All genotyping, breeding, handling and treatments in this study were done according to protocol and all animal studies were under an animal study protocol approved by the Institutional Animal Care and Use Committee of Albert Einstein College of Medicine.

### Cortical Primary Cultures

Cortical neurons were obtained from control (CTR) and L2AKO PO-P1 postnatal mice and neuronal cultures were prepared as follows: brain cortices were dissected and enzymatically digested (0.36 mg/ml papain in phosphate buffered saline (PBS) with D-glucose (6 mg/ml) and 1% bovine serum albumin for 15 minutes)37° C. Neurons collected by centrifugation, were resuspended in Neurobasal Medium (ThermoFisher 10888022), supplemented with 2% B27-Supplement (Gibco-Invitrogen, 17504044), 1% Penicillin/Streptomycin and 1% GlutaMAX (Fisher, 35050-061) and plated at a density of 2.5×10⁵ cells/cm² into 24-well Seahorse Bioscience plates (Agilent, 100777-004) pre-coated with CELL-TAK (CORNING, 354240) or in coverslips. The first 24 h the media contained fetal calf serum 15% (v/v) heat-inactivated. Cells in coverslips were co-stained with NeuN, GFAP and Hoechst to assess level of glial presence in the primary neuronal cultures.

### Cell lines

Mouse embryonic NIH3T3 fibroblasts from the American Type Culture Collection (ATCC) and mouse neuroblastoma CAD cell lines (gift from Dr. Duncan Wilson, Albert Einstein College of Medicine) were maintained in DMEM (Sigma-Aldrich) in the presence of 10% newborn calf serum (NCS) (Atlanta Biologicals). CAD cells were differentiated by serum removal and used at 5 days post-starvation. Lentivirus expressing the shRNA constructs against LAMP-2A and Atg7 were generated by the same protocol using the shRNA previously described.

### Antibodies

Primary antibodies were from the following sources (dilutions, commercial source and catalog number indicated in brackets): rabbit anti-LAMP-2A (1:5000, ThermoFisher Scientific, 512200), rabbit anti-LC3 (1:1000, Cell Signaling, 2775), rabbit anti-p62 (1:1000, Enzo Life Sciences, BMLPW98600100), mouse anti-β-actin (1:10000, Sigma, A4700), rat anti-LAMP1 (1:1000, Hybridoma Bank, 1D4B), rabbit anti-4HNE (1:1000, abcam, ab46545), rabbit anti-GFAP (1:1000, Dako, Z0334), rabbit anti-GFAP (1:1000, abcam, ab5804), rabbit anti-ubiquitin (1:1000, Dako, Z0458), rabbit anti-K48-ubiquitin (1:1000, Millipore, 05-1307), rabbit anti-K63-ubiquitin (1:1000, Millipore, 05-1308), mouse anti-Aβ (1:1000, Novus Biologicals, MOAB2-AF488), mouse anti-human-Tau (1:1000, Abcam, Tau13 ab 19030), mouse anti-Tau (HT7 clone) (1:1000, ThermoFisher Scientific, MN1000), mouse anti-phosphoTau Ser202-Thr205 (AT8 clone) (1:1000, ThermoFisher Scientific, MN1020), mouse anti-phosphoTau Ser422 (1:1000, generated in house using the phosphopeptide CSIDMVD-pS-PQLATLAD as antigen (Grueninger et al., 2010)), rabbit anti-GATE16 (1:1000, MBL, PM038), mouse anti-misconformed tau (MC1) (1:1000, a generous gift from Dr. Peter Davis), rabbit anti-Clathrin (Clone D3C6—1:1000—Cell Signaling 4796S), mouse anti-AP2α (Clone 3B5—1:1000—ThermoFisher Scientific, MA1-872), rabbit anti-Arpc2 (1:1000—Novus Biological NBP188852), mouse anti-APP (1:1000—Biolegend 802803), mouse anti-PDH (1:1000—ThermoFisher Scientific, 459400), rabbit anti-CathepsinD (CathD—1:1000—Abcam #ab75852), rabbit anti-GBA (1:1000—Sigma Aldrich, G4171), mouse anti-Hsc70 (1:1000—Novus Biological NB120-2788), rat anti-Hsp90 (1:1000—Stressgen ADI-SPA-835-F), rabbit anti-Hsp40 (1:1000—Stressgen ADI-SPA-400), mouse anti-Rac1 (Clone23A8—1:1000—Millipore 05-389), rabbit anti-Phlpp1 (1:1000—Bethyl A300-660A), anti-Rictor (1:1000—Bethyl A300-459A), anti-CathepsinA (Ctsa—1:1000—Abcam #ab184553), rabbit anti-RARα (1:1000—Cell Signaling 2554), mouse anti-CaMKIIα (1:200—Millipore #05-532). All the secondary antibodies were from ThermoFisher Scientific. All antibodies used in this study were from commercial sources and were validated following the multiple dilution method and, where available, using cell lines or tissues from animals knock-out for the antigen.

### Behavioral Procedures

All behavioral procedures were performed by investigators blind to genotype of each group or nature of intervention. To decrease stress related to procedures, all animals were first habituated to handling by the experimenter and to the procedure room for at least 1 hour prior to testing. Limb Clasping. Clasping was assessed for 5 seconds and scored. Spontaneous alternation in a Y-maze. Mice were allowed to freely explore the maze for 10 min. Number and order of arm entries were quantified. Alternation index was calculated as. Stride length. Stride length was measured in a 4.5 cm×40 cm corridor following inking of hindlimb paws. The three longest stride lengths (corresponding to maximum velocity) were considered. Short-term memory test in Y-maze. Test was performed in a Y-shaped maze with three arms angled at 120°. Visual cues were placed on surrounding walls. On the first trial (learning), animals explored the maze for 8 minutes with only two arms opened (‘start’ and ‘familiar’ arms). Access to the third arm (‘novel’) was blocked by an opaque door. After a 1 hour retention time, mice were placed again in the maze for 5 minutes with all arms accessible (test). Exploration was recorded and an automated tracking system was used. Data are reported as fraction of time spent in the novel arm. Negative geotaxis. Mice are placed on the sloped platform (50°) facing in a downward direction. The latency to turn and orient themselves to be facing up the slope was recorded. Novel object recognition. Novel object recognition was performed after training mice in an open field arena with identical objects for 4 minutes, followed by 2 hours retention time. Mice were placed in the same arena after replacing one of the familiar objects by a novel object and exploration of both objects was quantified for 4 minutes. Novelty preference is quantified as amount of time dedicated to the exploration of the novel object. Discrimination index is the difference between the exploration time of the novel and familiar object over the total exploration time. Elevated plus maze. Anxiety-like behavior was quantified as follows. Briefly, mice were allowed to freely explore an elevated plus maze with two open arms and two closed arms. Quantification of the % of time spent to explore the open arms versus the closed arms was done. Forced Swim test. Mice were placed in cylinder tank (30 cm×20 cm) filled with water at room temperature. Animals were gently placed in water and immobility was quantified over a total time of 9 minutes. Open field. Mice were allowed to freely move in an open field arena (50×50 cm) for 10 min. Tracking was performed using ezTrack (Pennington et al., 2019). The number of animals selected for each behavior test was determined by power analysis. In those cases, in which test allowed for repetition without risking a co-funding effect of “learning the test” or where multiple tests in the same animal were possible, we performed testing in higher number of animals than the minimal determined by power analysis in order to further strengthen confidence in the findings.

### Tissue Dissection and Histological Procedures

Mice were euthanized with pentobarbital overdose (100 mg/kg i.p.) and intracardially perfused with 0.9% saline solution. Brains were removed quickly after death. Each brain was then dissected along the midline. The right hemisphere was post-fixed overnight in 4% paraformaldehyde, cryoprotected in PBS containing 20% sucrose before being freeze by immersion in a cold isopentane bath (−50° C.), and stored immediately at −80° C. until sectioning. Brains were sectioned in a Leica CM3050S cryostat (Leica Microsystem, Wetzlar, Germany) at −20° C. in either coronal or sagittal 40 μm-thick free-floating sections and stored in PBS containing 0.2% sodium azide at 4° C. until use. The left hemisphere was dissected, and several brain regions were collected for further analysis: cortex, hippocampus, midbrain, striatum and cerebellum. Samples were stored at −80° C. until use. Prior to staining, sections of appropriate levels (e.g. striatum, midbrain or hippocampus) were selected. Immunostainings were performed as follows. Briefly, selected sections were washed, incubated in blocking buffer, and then incubated overnight with the appropriate primary antibody. The following day, sections were washed 3 times for 5 minutes in PBS, incubated with the appropriate anti-specie secondary antibody (1:2000), washed 3 times in PBS and mounted. Cell nuclei were stained using Hoechst (Life Technologies, 33342) at 1:5,000 for 30 sec prior to mounting. ProLong Diamond mounting medium was used (ThermoFisher Scientific P36965). For the immunostainings in the Tg and Tg-L2A^{-/-} immunofluorescent detection of phosphorylated tau aggregates and amyloid plaques was performed. The amyloid-specific BAP-2 antibody was replaced by MOAB2-AF488 (Novus Biologicals). Images were acquired with an Axiovert 200 fluorescence microscope (Carl Zeiss Microscopy), or when full brain sections were imaged individual images from the scanning of brain slices were mounted with an ApoTome.2 slider, or a Leica confocal TCS-SP8 (Leica Microsystem) and prepared using ImageJ Software (NIH). A perceptually uniform lookup table (Magma) was used to enhance contrast and highlight pattern and intensity differences between experimental groups.

### Thioflavin S Staining

Thioflavin S staining was performed prior to incubation in the blocking buffer using a 0.5% Thioflavin S solution (Santa Cruz, sc391005) in water for 7 minutes at room temperature.

### Histopathology of Peripheral Organs and Blood Cell Count

Where indicated mouse liver, lung and kidneys were dissected and fixed in 1% PFA overnight and paraffin embedded. Tissues were sectioned, stained with hematoxylin and eosin (H&E), and analyzed by an expert pathologist, blind to the treatment groups, to score for possible presence of toxicity in these organs. Blood cell count in the groups of mice administered vehicle or CA was analyzed in tail blood drawn monthly and at the moment of tissue dissection using an Oxford Science Forcyte Blood Analysis Unit.

### Western Blotting

Protein concentration was determined using the Lowry method with bovine serum albumin as a standard (Lowry et al., 1951). Dissected brain regions were solubilized on ice with RIPA buffer (1% Triton X-100, 1% sodium deoxycholate, 0.1% SDS, 0.15M NaCl, 0.01M sodium phosphate, pH7.2) followed by sonication. Immunoblotting was performed after transferring SDS-PAGE gels to nitrocellulose membrane and blocking with 5% low-fat in milk 0.01% Tween-TBS for 1 h at room temperature. The proteins of interest were visualized after incubation with primaries by chemiluminescence using peroxidase-conjugated secondary antibodies in LAS-3000 Imaging System (Fujifilm, Tokyo, Japan). Densitometric quantification of the immunoblotted membranes was performed using ImageJ (NIH). All protein quantifications were done upon normalization of protein levels to a loading control (β-actin) or Ponceau staining and expressed as fold of the relevant control group.

### Autophagy Measurements in Cultured Cells

Macroautophagic flux was measured in protein lysates using immunoblot for LC3-II in cells treated or not with lysosomal protease inhibitors (20 mM ammonium chloride and 100 μM leupeptin). Flux was calculated as the increase in levels of LC3-II in protease inhibitors-treated cells relative to untreated cells. CMA activity was measured in cells stably transduced with lentivirus carrying the KFERQ-PS-Dendra reporter and plated in glass-bottom 96-well. Sixteen hours after photo switching with a LED lamp (405 nm for 3 minutes), cells were fixed with 4% PFA and imaged using high-content microscopy (Operetta system, Perkin Elmer). Images were quantified using the manufacturer's software in a minimum of 800 cells.

### Transferrin Uptake in Differentiated CAD Cells

Transferrin internalization was performed as previously. Briefly, CAD cells grown in serum-free DMEM were incubated for 10min using Alexa555-conjugated transferrin (25 μg/ml; Life Technologies). The cells were then transferred on ice and wash 3 times with ice-cold PBS. Cells were then fixed for immunofluorescence.

### Isolation of Sarkosyl Insoluble Fraction

Brain homogenates were prepared as described in. Homogenates from several mice of each genotype were pooled and diluted to a final protein concentration of 1 mg/ml. Sarkosyl was then added to a final concentration of 1% and the homogenates incubated for 30 min at 4° C. The homogenates were subsequently centrifuged at 100,000× g for 1 hr. Pelleted proteins were sent for proteomic analysis or were resuspended directly in SDS-PAGE sample buffer and boiled for 2 min. For each genotype, equal volumes of resuspended pellet were used for SDS-PAGE/western blotting.

### AlphaLISA Immunoassays

Tau, pS422-Tau, pS202/T205-Tau, and aggregated tau were measured by immunoassay in brain extracts as described in (Grueninger et al., 2010), except that the MSD assay format was replaced by AlphaLISA immunoassay technology (Perkin-Elmer). Aβ42 Enzyme-Linked Immunosorbent Assay (ELISA)

Aβ42 levels were measured by ELISA using a commercial kit (ThermoFisher #KHB3442). Mouse brain lysates were diluted 1:50 in the provided diluent and assay was performed following manufacturer's recommendations.

### Extracellular Flux Analysis

Oxygen consumption rates and extracellular acidification rates were measured using a 24-well Seahorse Bioanalyzer XF 24 according to the manufacturer's instructions (Agilent Technologies). Briefly, neurons were plated into 24-well plates pre-coated with CELL-TAK (CORNING, 354240at a concentration of 1.8×10¹⁴ cells/well and used at 14 days-in-vitro. Once in the reader, plates were sequentially injected 10 mM glucose, 1.0 μM oligomycin and 50 mM 2-Deoxyglucose (2-DG) in artificial cerebrospinal fluid (aCSF, 120 mM NaCl, 3.5 mM KCl, 1.3 mM CaCl₂, 0.4 mM KH₂PO₄, 1 mM MgCl₂, 5 mM HEPES) +2 mM glutamine, pH: 7.4. Differentiated CAD cells plated on gelatin-coated plates were switched to manufacture provided base medium supplemented with 2 mM L-glutamine and sequentially injected with 10 mM glucose, 2 mM oligomycin and 100 mM 2DG in the bioanalyzer. Quantifications were performed using Seahorse Wave Desktop software. Data were normalized to cell number using CyQuant (ThermoFisher, C7026).

### Pharmacokinetic Properties

ICR (CD-1) male mice were fasted at least three hours and water was available ad libitum before the study. Animals were housed in a controlled environment, target conditions: temperature 18 to 29° C., relative humidity 30 to 70%. Temperature and relative humidity were monitored daily. An electronic time-controlled lighting system was used to provide a 12 hr light/12 hr dark cycle. 3 mice for each indicated time point were administered 10 mg/Kg CA77.1 by oral gavage or 1 mg/Kg by intravenous injection using 30% PEG-400, 65% D5W (5% dextrose in water), 5% Tween-80 vehicle. Mice were sacrificed, and brain samples were harvested at 0 hr, 0.25 hr, 0.5 hr, 1 hr, 2 hr, 4 hr, 8 hr, 24 hr, and analyzed for CA77.1 levels using LC-MS/MS. Pharmacokinetics parameters were calculated using Phoenix WinNonlin 6.3. Experiments performed at SIMM-SERVIER joint Biopharmacy Laboratory.

### Quantitative Proteomics and Protein Pathway Analysis Insoluble Fractions of Autophagy-Deficient Mice

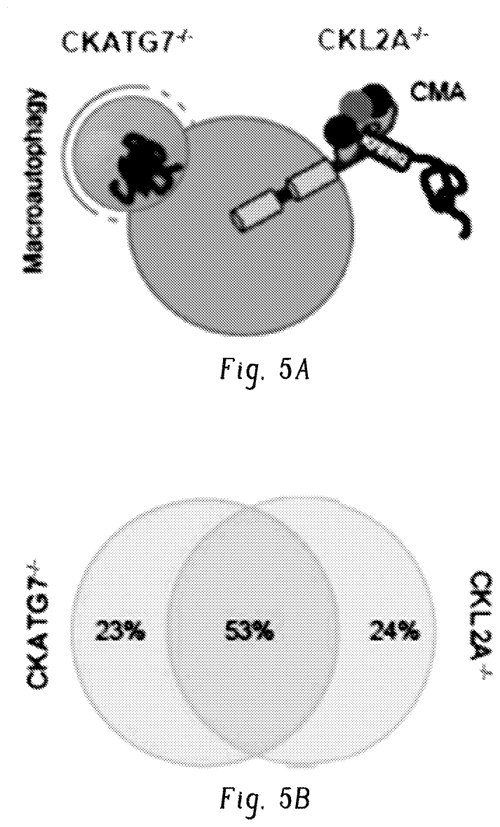

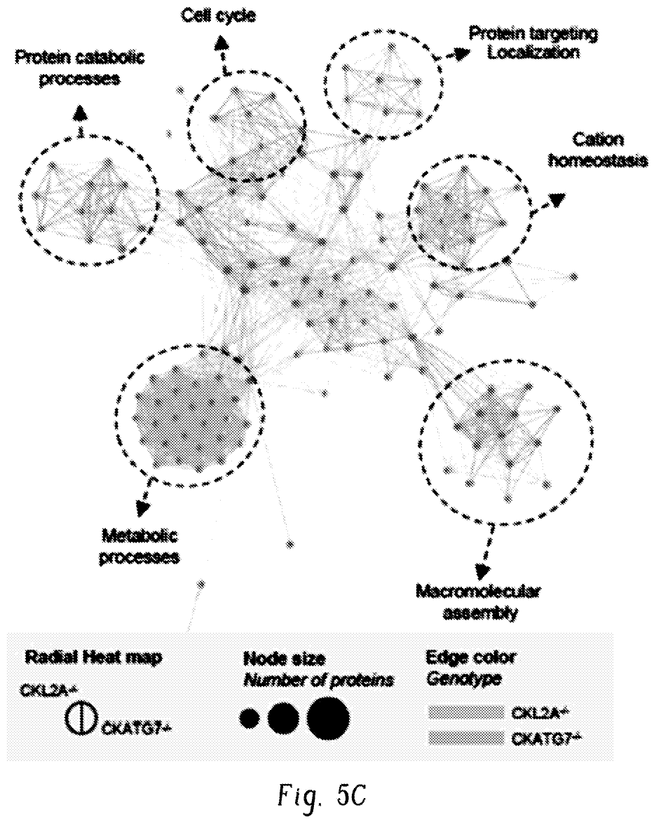

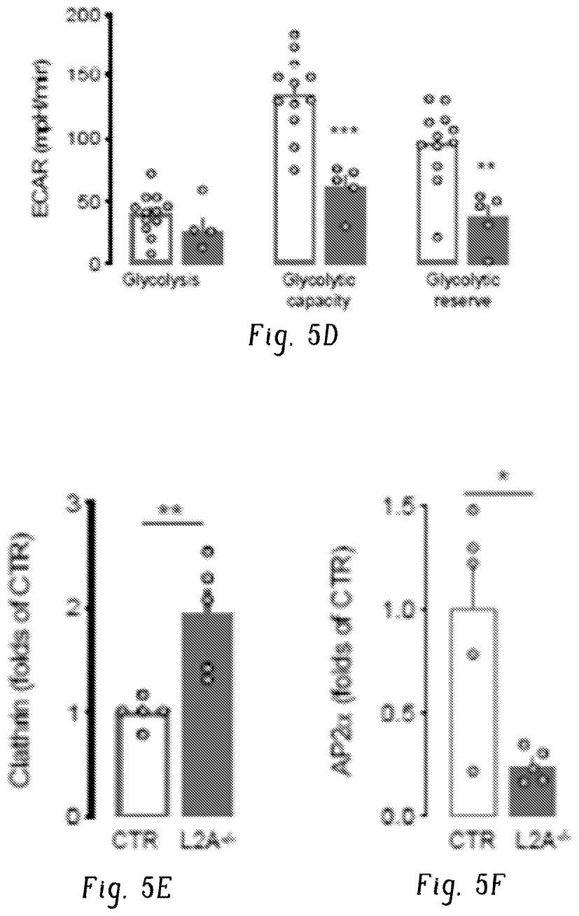

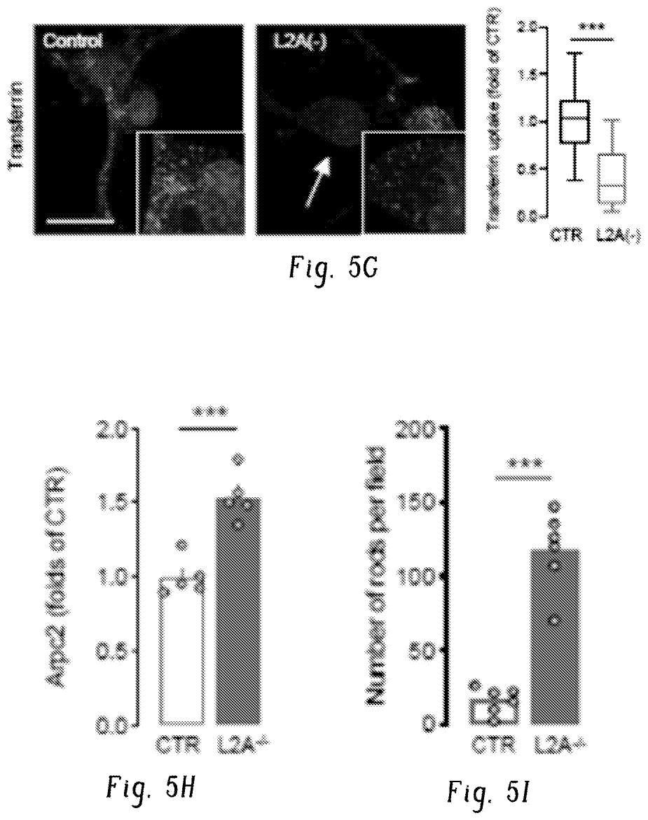

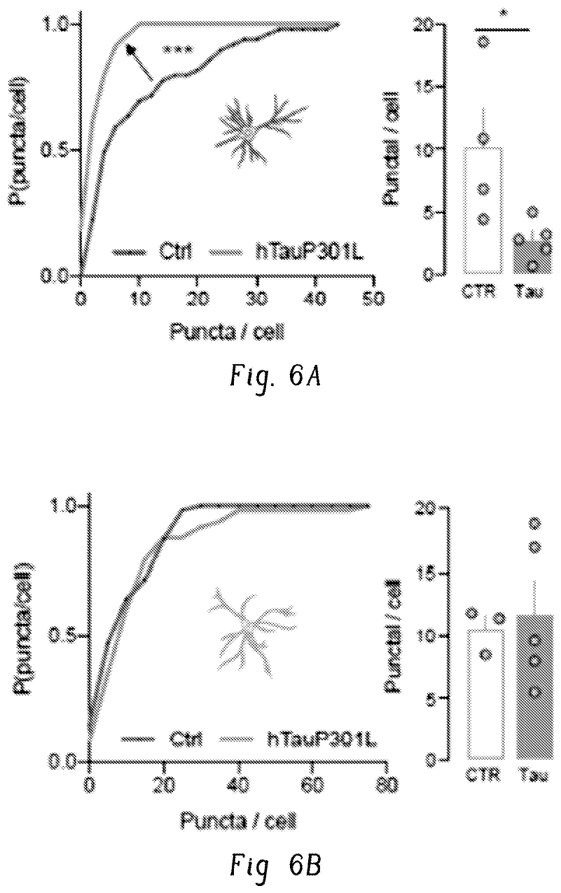

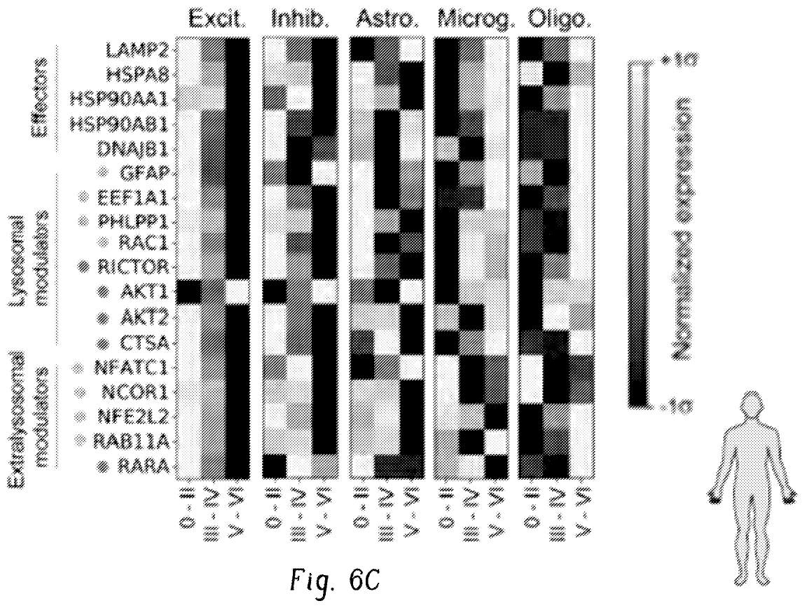

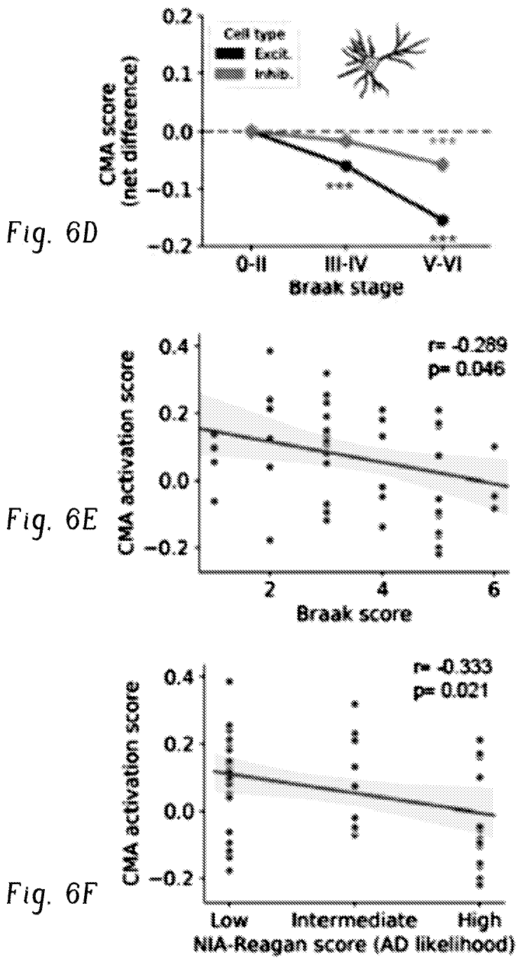

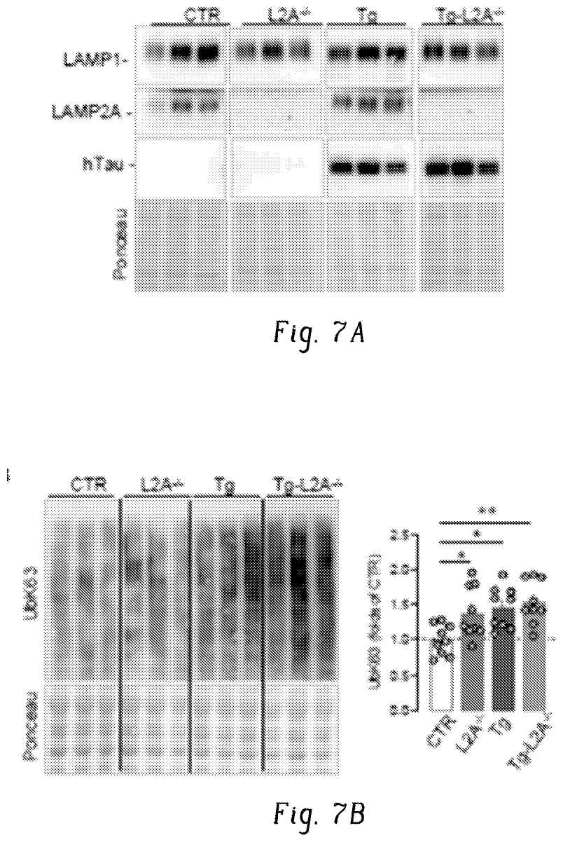

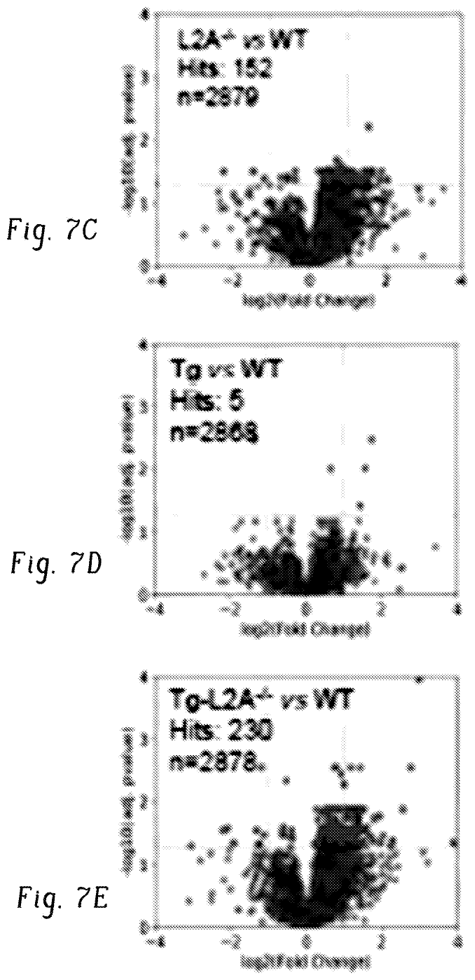

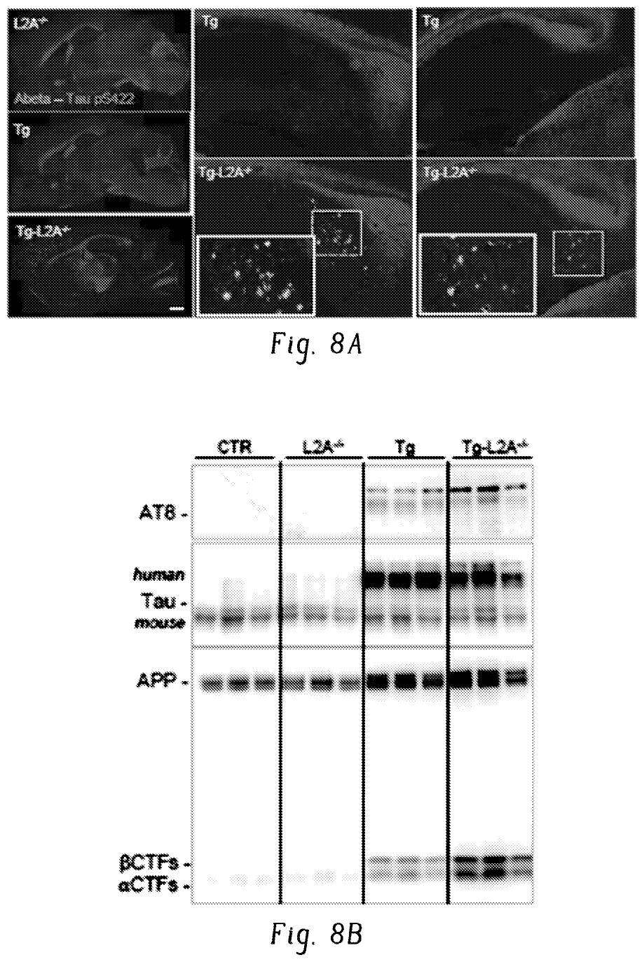

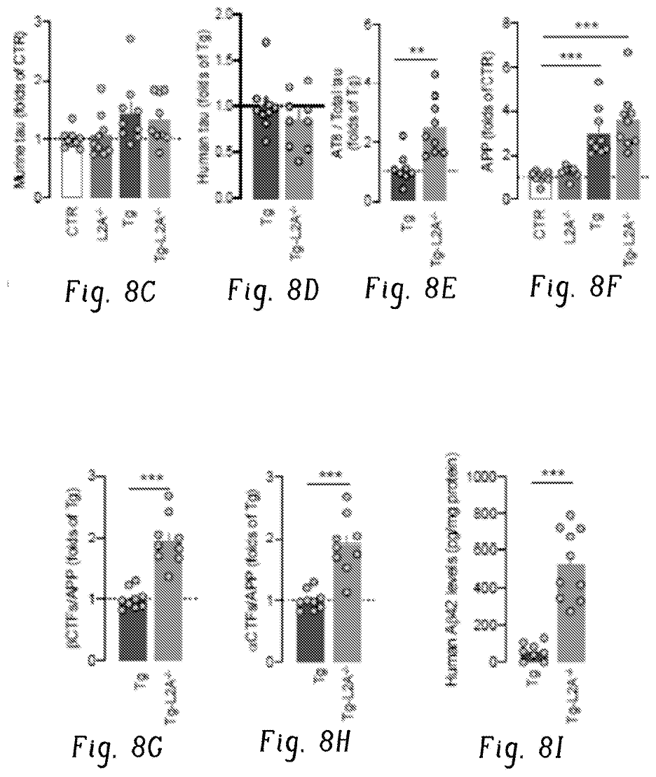

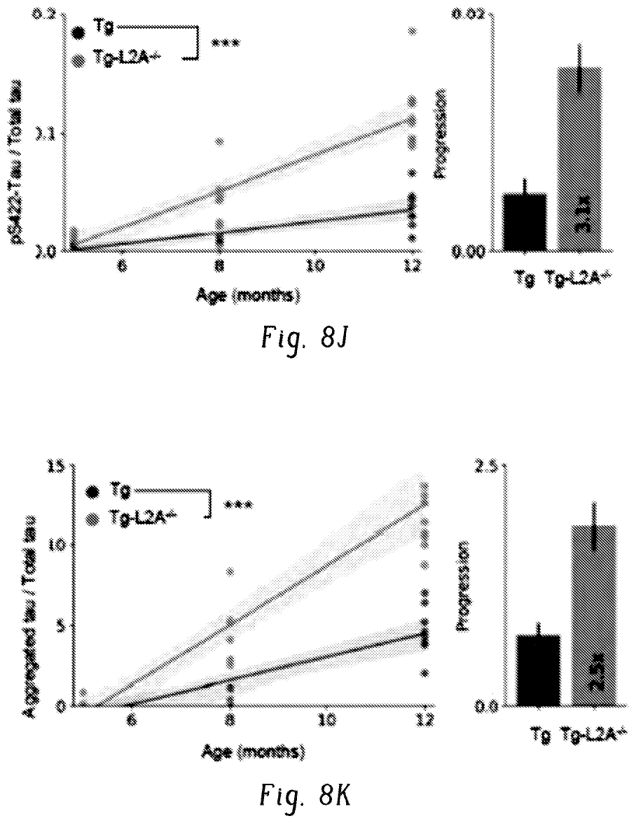

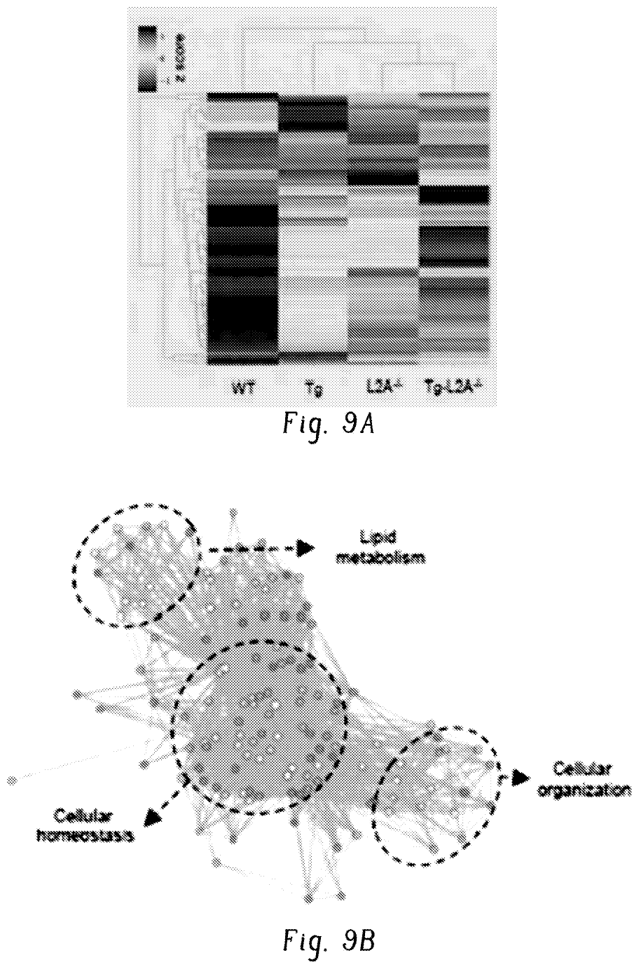

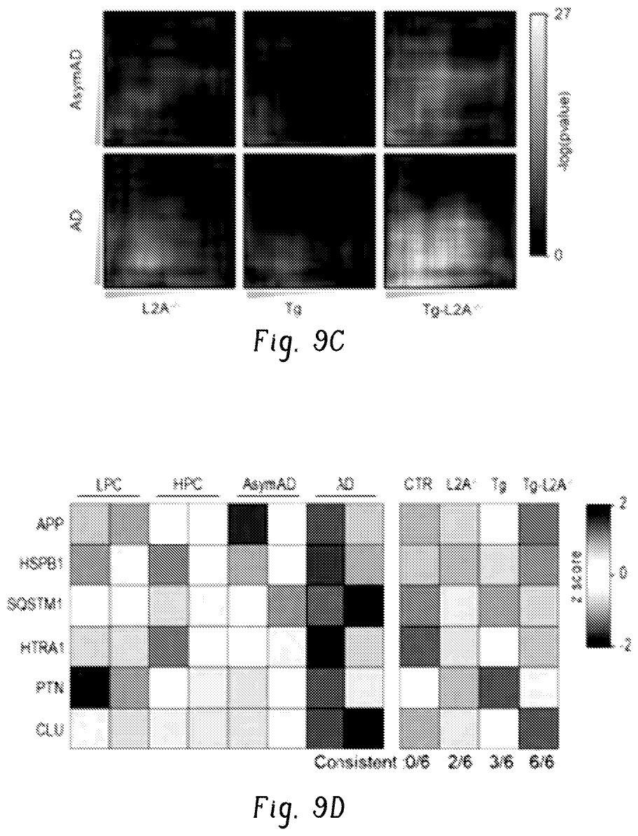

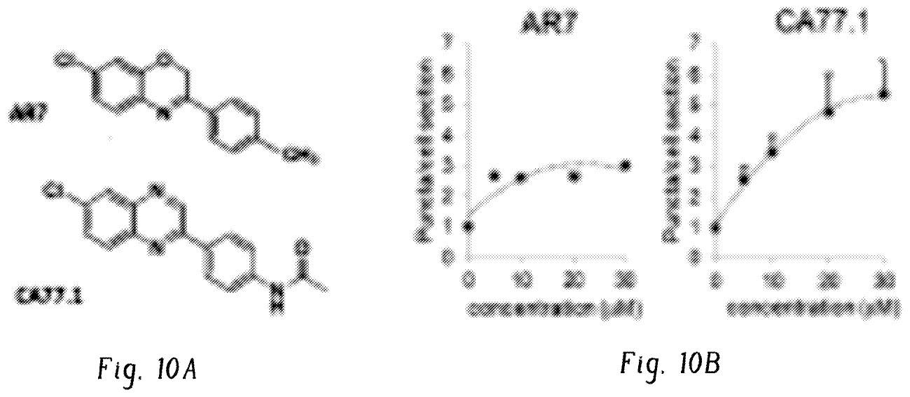

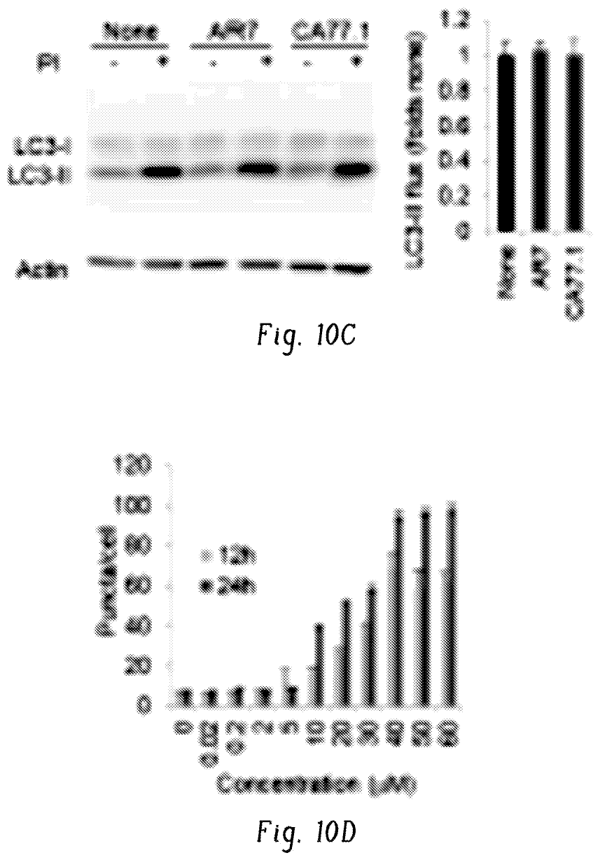

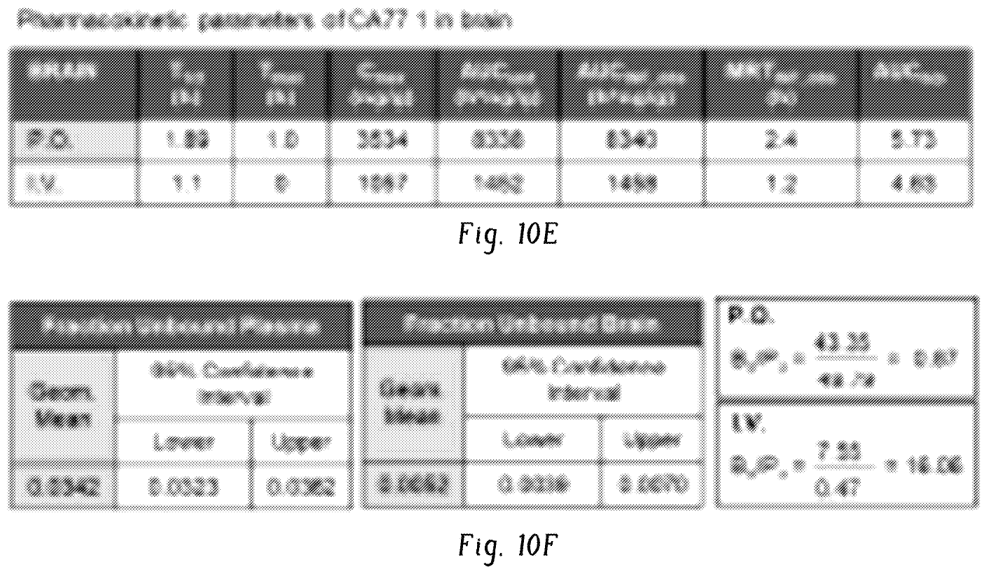

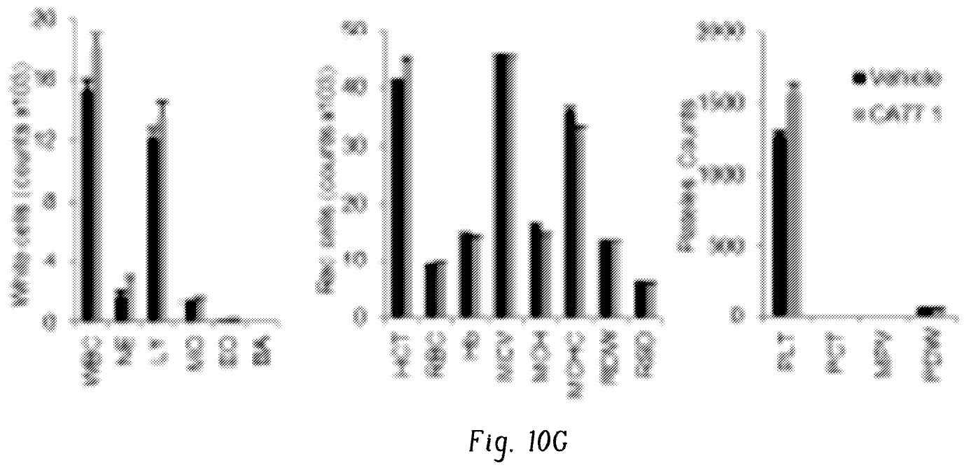

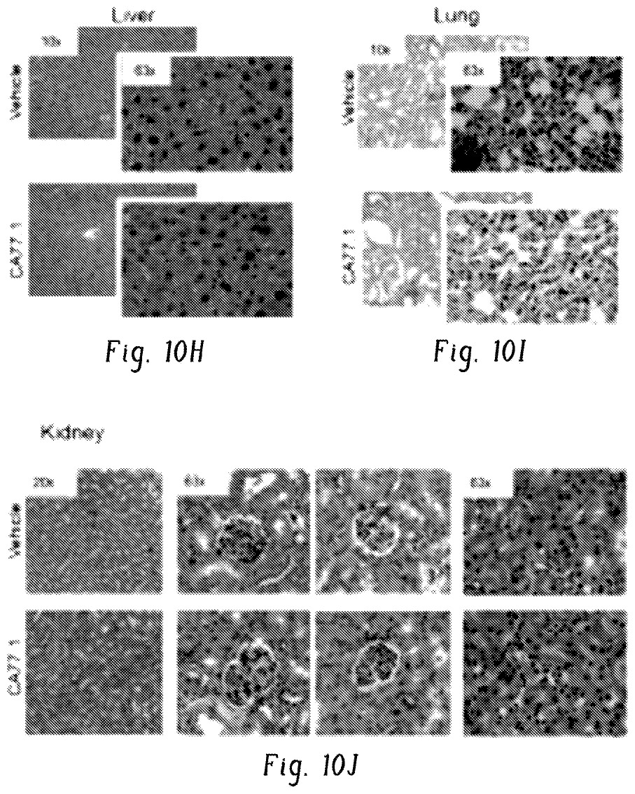

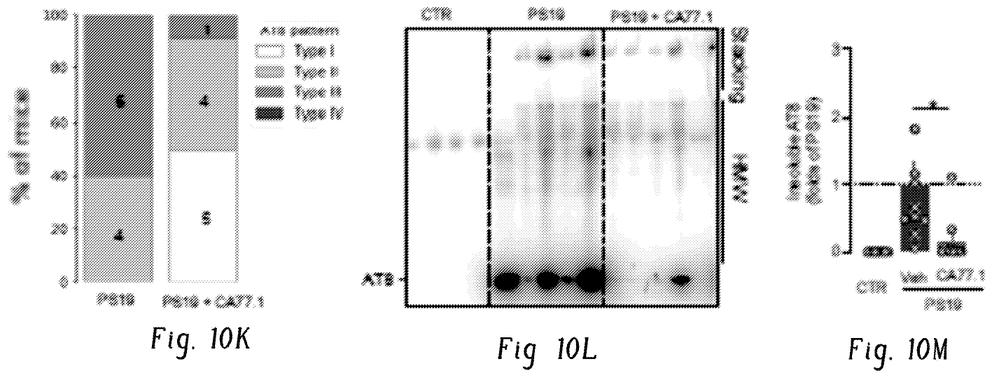

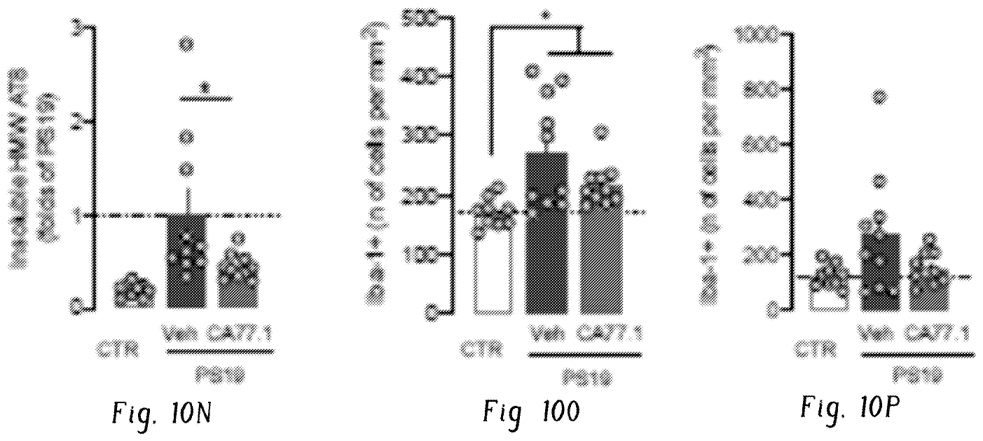

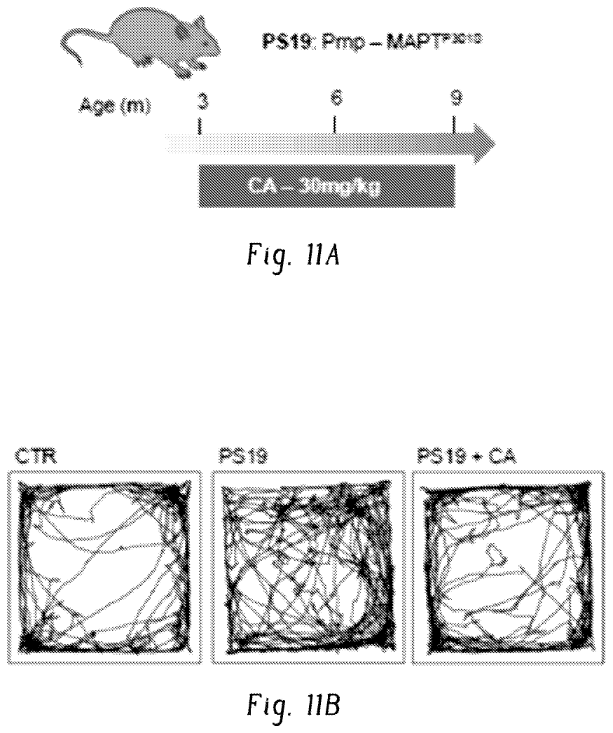

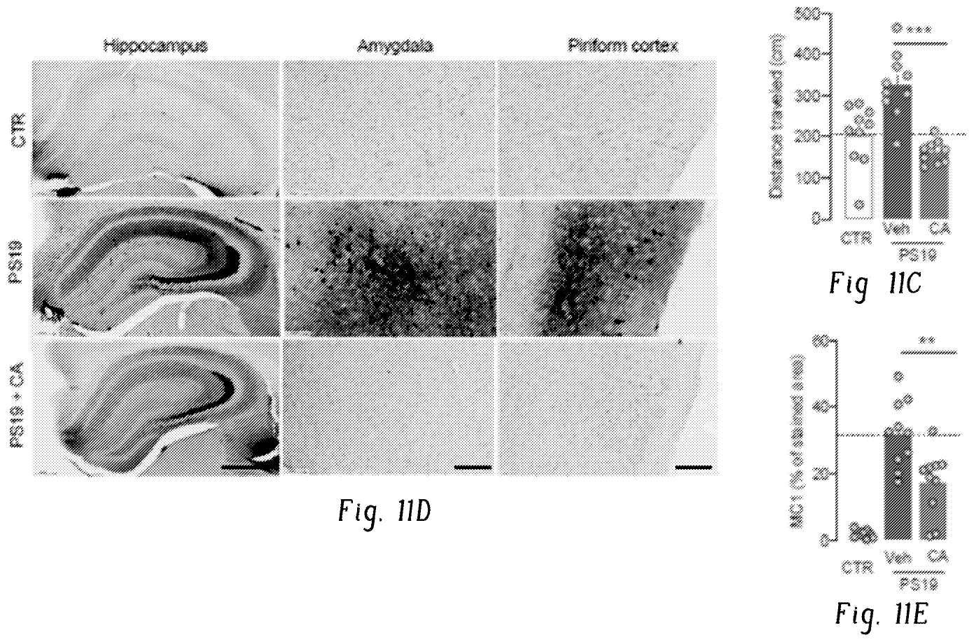

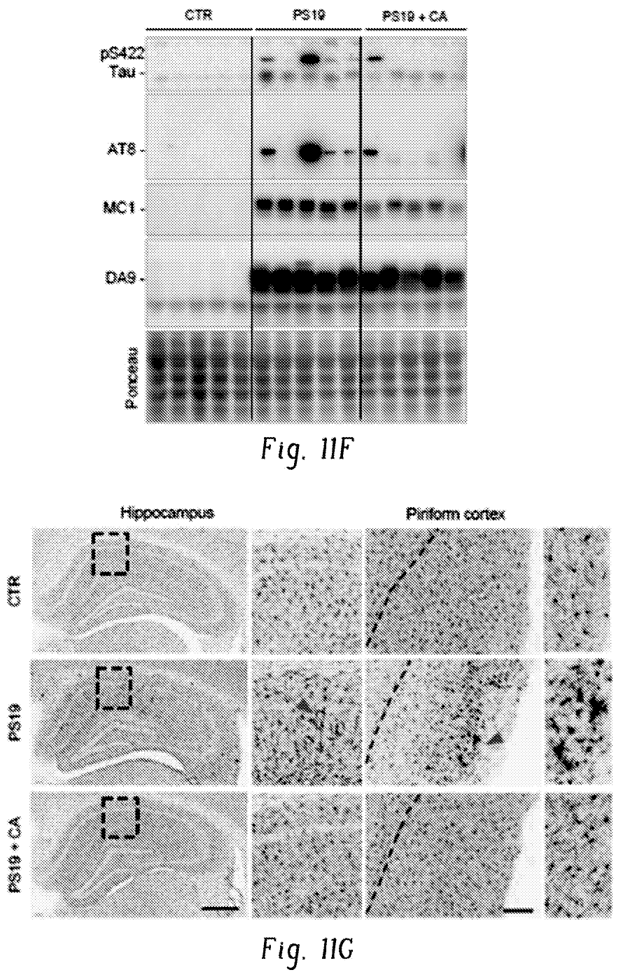

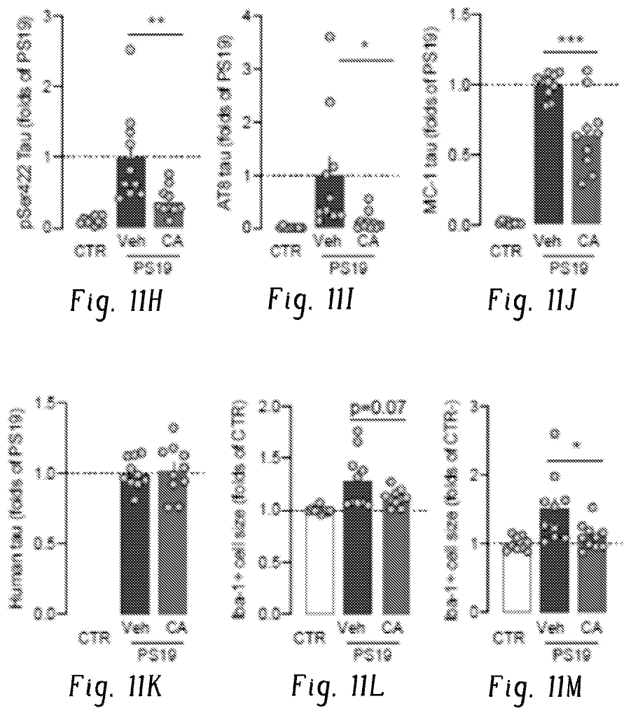

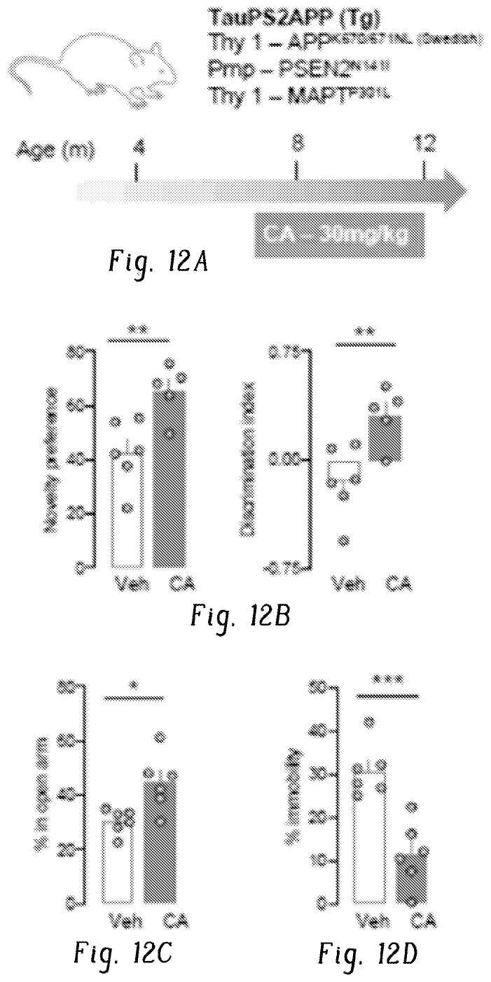

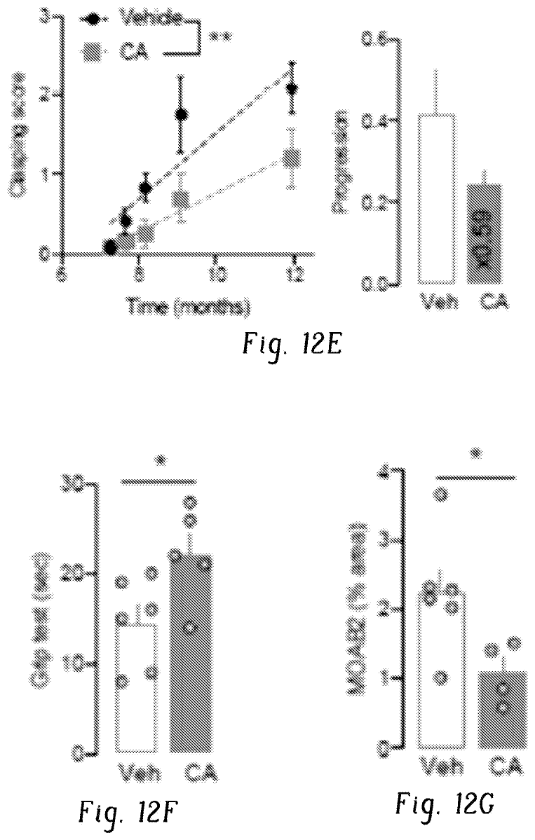

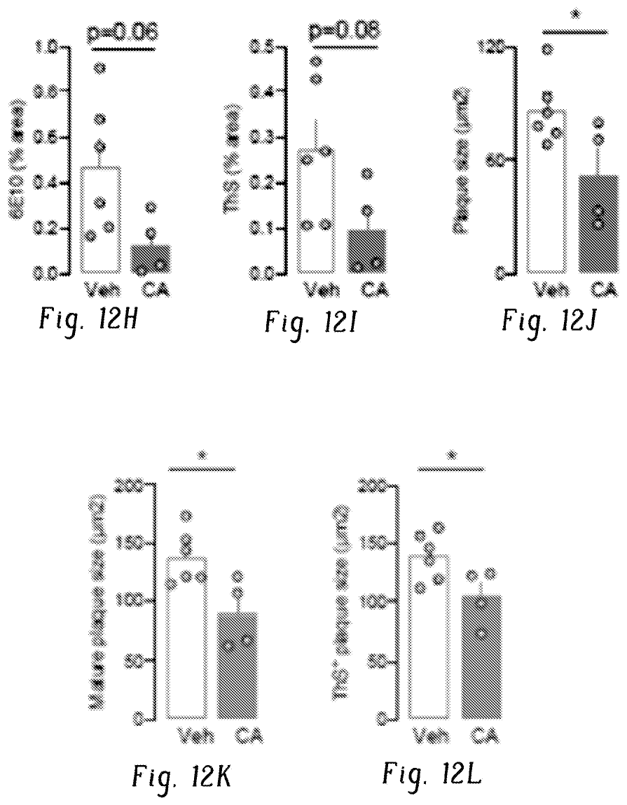

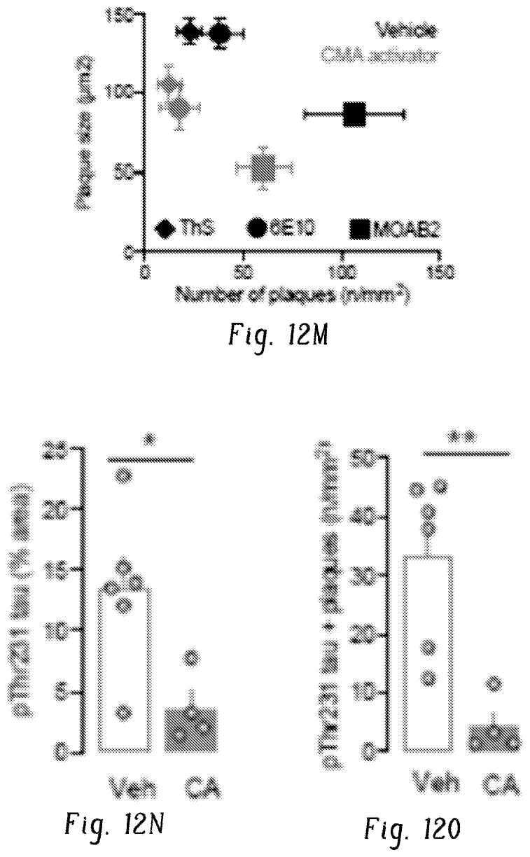

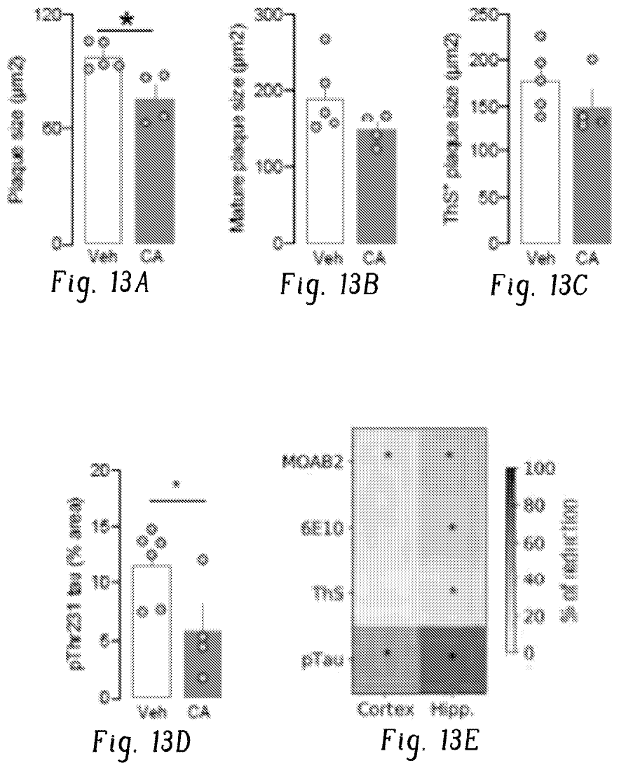

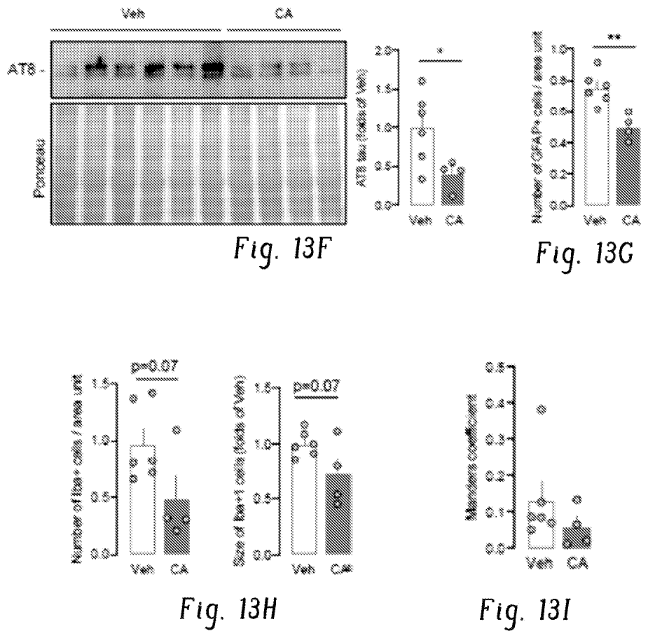

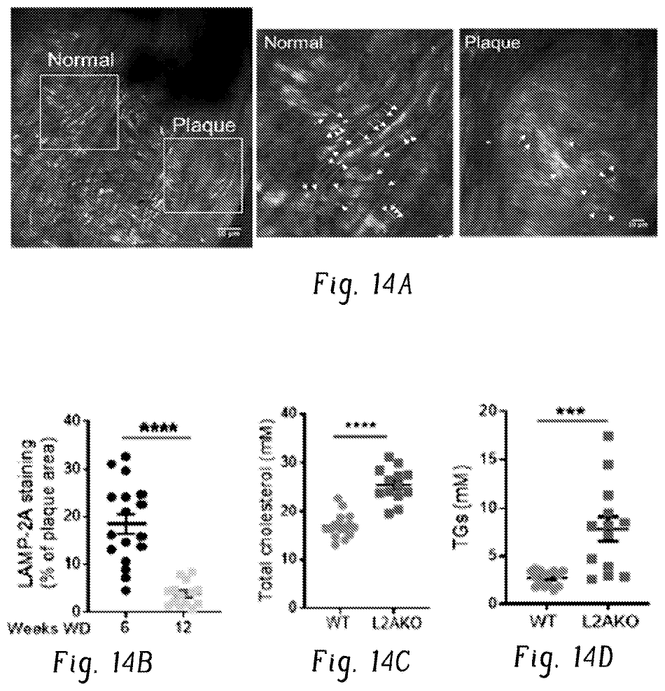

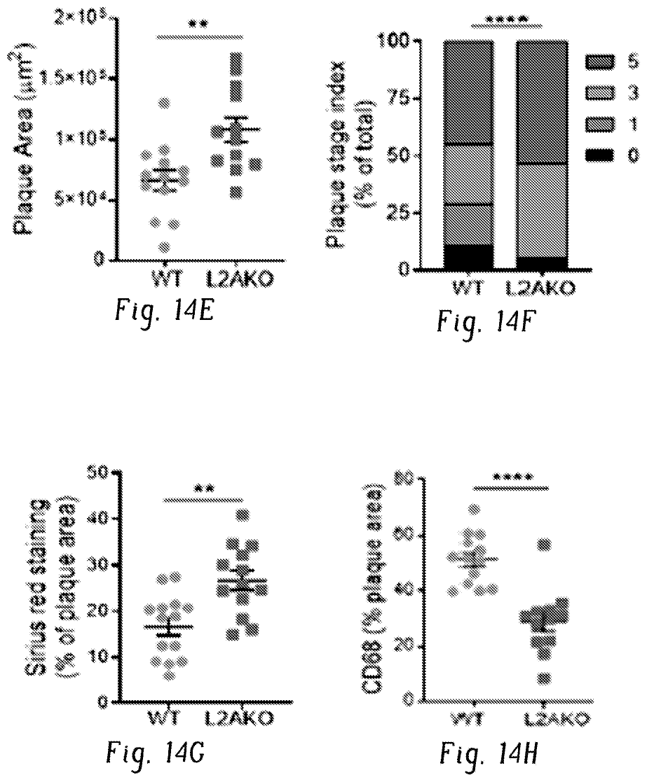

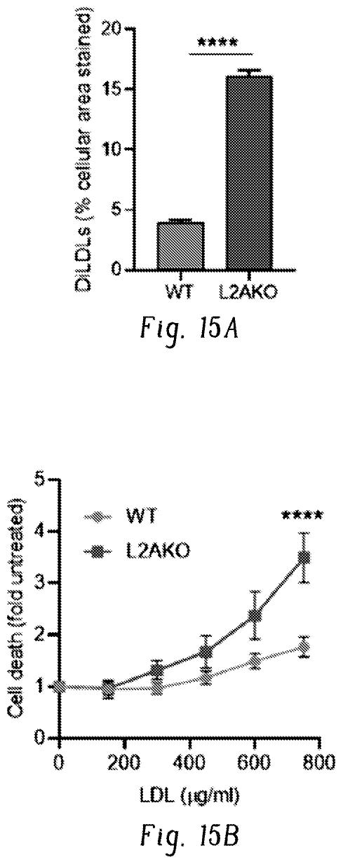

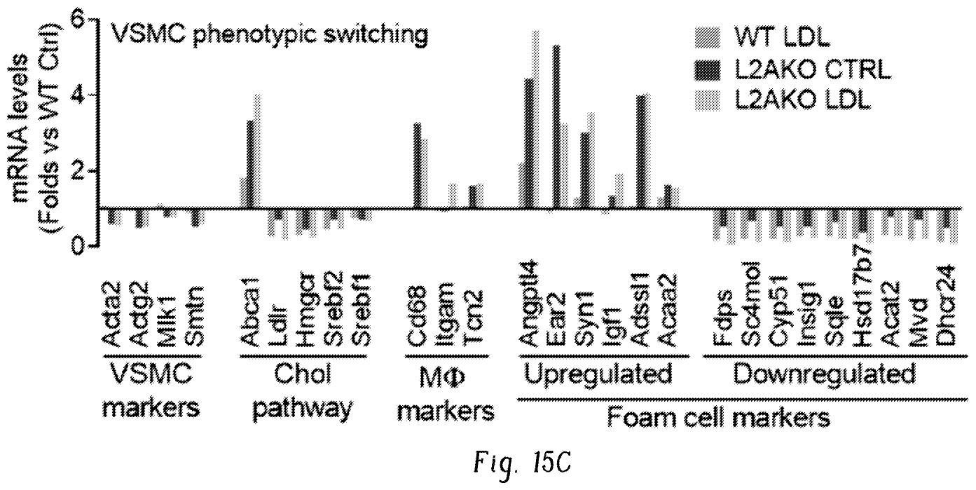

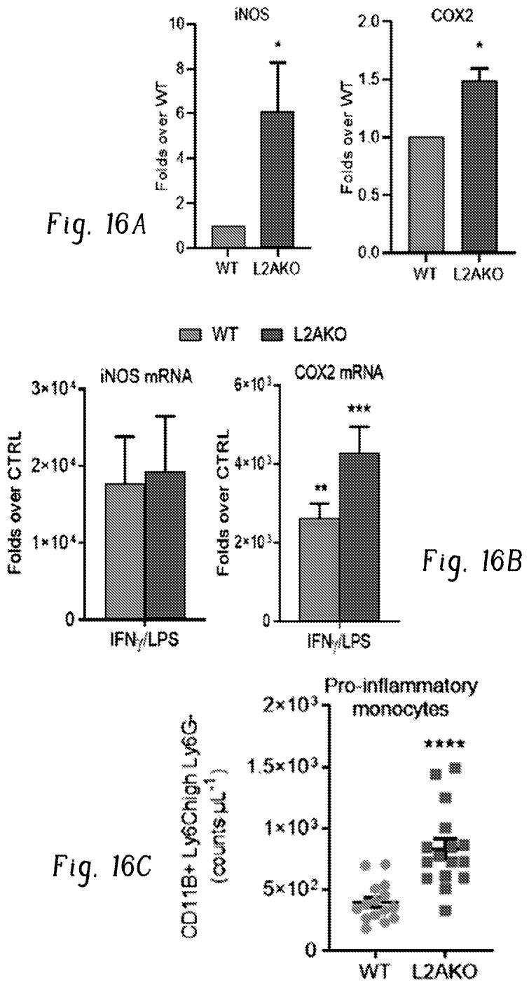

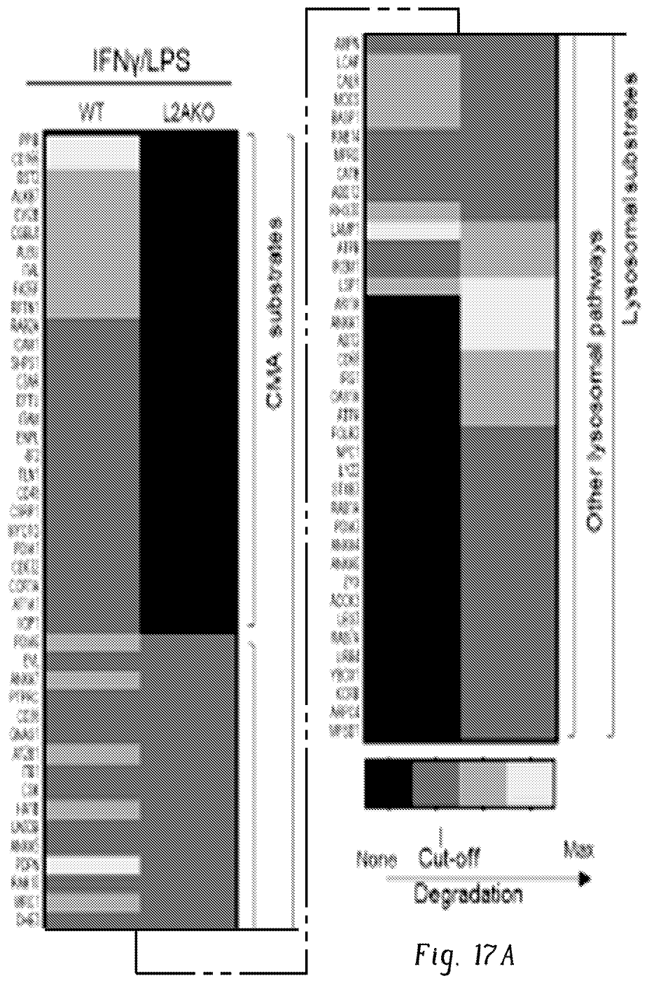

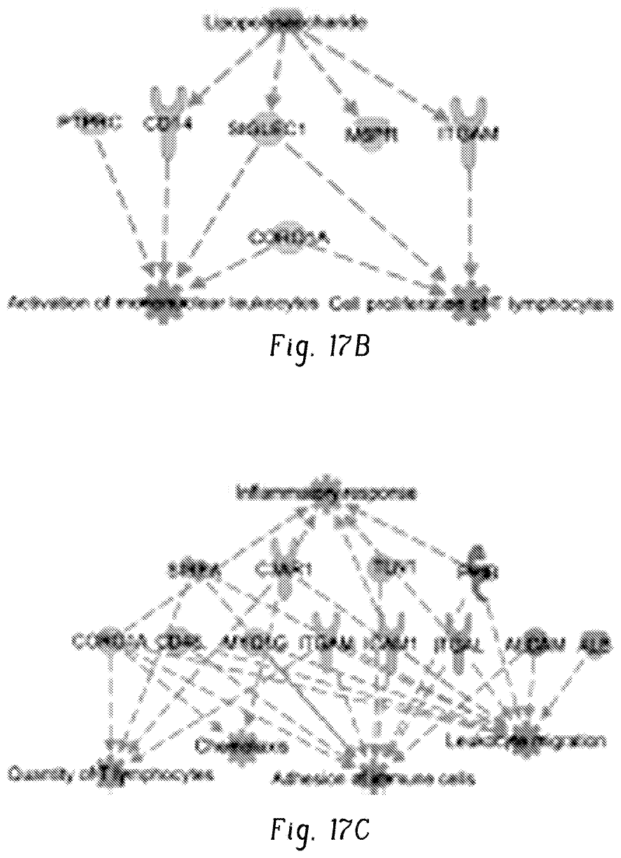

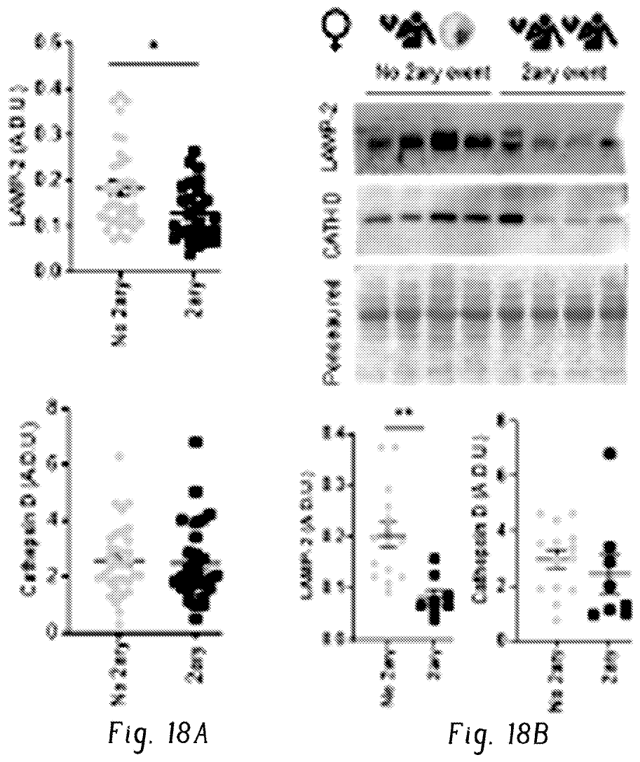

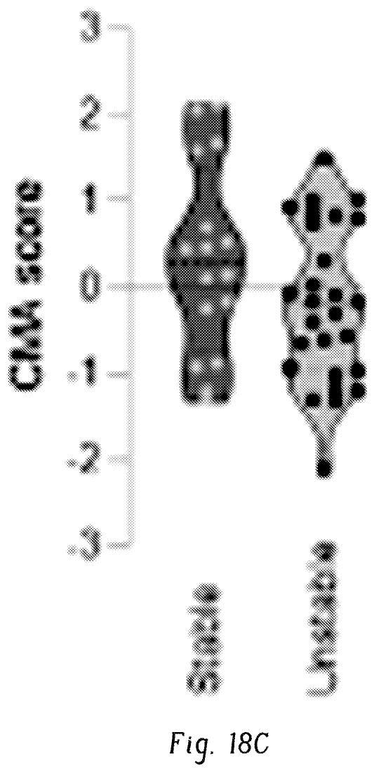

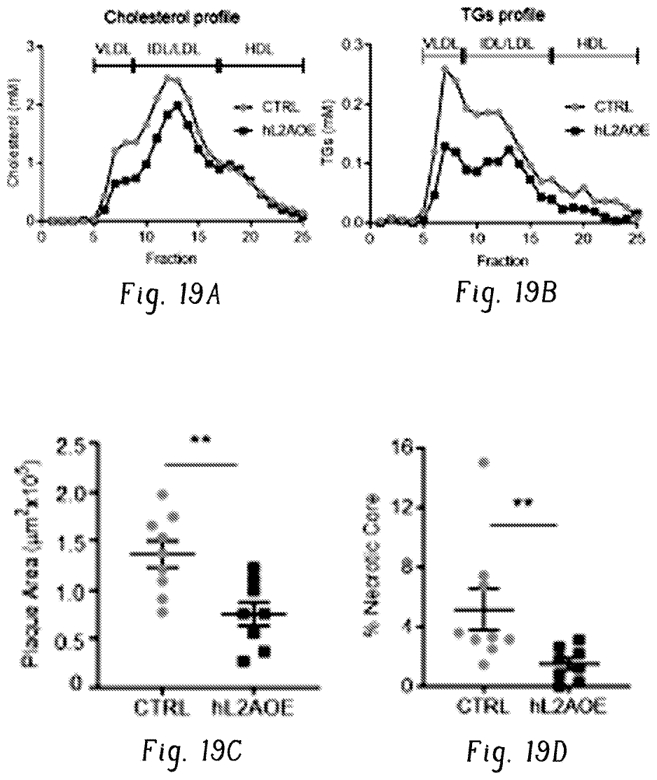

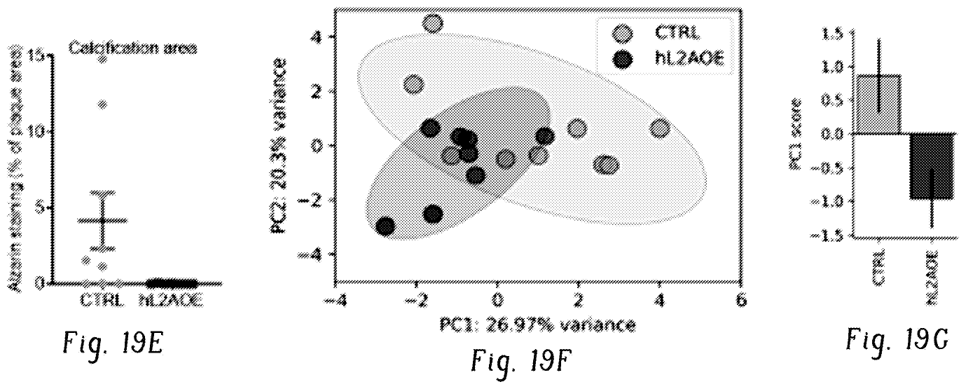

Sarkosyl-insoluble fractions from three different animals per genotype (Ctr, CKL2A^{-/-}, and CKATG7^{-/-}) were pooled. Quantitative proteomic analysis was performed using iTRAQ multiplex (Applied Biomics). For each protein hit the average ratio(s) for the protein, the number of peptide ratios that contributed and the geometric standard deviation were determined. Values in the experimental groups were compared to Ctr and are represented as folds. For the comparative analysis between the sarkosyl-insoluble fractions of CKL2A^{-/-} and CKATG7^{-/-} mice (FIG. 7), we first selected proteins with a significant enrichment over that in the same fraction in CTR mice (as presented in FIG. 5). This allowed us to exclude the large functional multi-protein complexes and membrane-associated structures detectable in the sarkosyl-insoluble fraction that are not relevant to protein aggregation.

### Alzheimer's Disease—CMA Deficient Mice

Protein was precipitated from lysates (cortex and hippocampus pooled) from three mice of each genotype (WT, L2A^{-/-}, Tg, Tg-L2A^{-/-}), solubilized in 8M urea, 0.1 M ammonium bicarbonate pH 8.0, 150 mM NaCl, complete mini protease and phosphatase inhibitors (Roche) and cysteine residues were reduced and alkylated with TCEP and iodoacetamide, followed by a 5-fold dilution with 0.1M ammonium bicarbonate. Proteins were digested into peptides by the addition of trypsin over night at 37° C. (1 μg trypsin per 100 μg lysate). Samples were desalted on C18 cartridges (NEST), lyophilized, resuspended in 4% formic acid, 3% acetonitrile and approximately lug of digested peptides per sample were loaded onto a 75 μm ID column packed with 25 cm of Reprosil C18 1.9 μm, 120 Å particles (Dr. Maisch GmbH HPLC, Germany). Peptides were eluted into an Orbitrap Fusion Lumos Tribrid mass spectrometer (Thermo Fisher Scientific) over the course of a 120 minute acquisition by gradient elution delivered by an Easy 1200 nLC system (Thermo Fisher Scientific). The composition of mobile phase A and B were 0.1% formic acid in water and 0.1% formic acid in 80% acetonitrile, respectively. All MS spectra were collected with orbitrap detection and the most abundant ions were fragmented by higher energy collision dissociation and detected in the ion trap, with a 3 second cycle time between MS1 spectra. All data were searched against the Uniprot mouse database (downloaded Jul. 19, 2016). Peptide and protein identification searches were performed using the MaxQuant data analysis algorithm, and all peptide and protein identifications were filtered to a 1% false-discovery rate. Label-free quantification and statistical testing was performed using the MSstats statistical R-package. Significantly modified proteins were selected by p<0.05 followed correction using the Benjamini-Hochberg protocol (FDR 5%). When FDR correction led to no hit, inspection of uncorrected p-values distribution was performed: if an anti-conservative distribution was observed, we applied alternative method of false discovery rate control by lowering threshold for significance (p<0.01) and using fold change cutoff (|fold change|>1.25), as previously suggested. The mass spectrometry data files (raw and search results) have been deposited to the ProteomeXchange Consortium (http://proteomecentral.proteomexchange.org) via the PRIDE partner repository with dataset identifier PXD017108.

### Rank-Rank Hypergeometric Overlap Test

Relevant protein lists were ranked according to fold change compared to Ctr mice and submitted to rank-rank hypergeometric overlap test in R (v. 3.6.2) using default settings. Colormap indicates -log₁₀ p value of the exact Fisher test by bin. We also used proteome-wide transition mapping, that consists of serialized differential expression analysis between genotypes using overlap quantification by RRHO and has the advantage of being threshold free and allowing a statistical assessment of the overlap extent. In our studies with Tg, Tg-L2A^{-/-} and L2A^{-/-} mice, we used comparison with this method of a fold change-ranked list of proteins of each genotype (normalized to CTR).

### Gene-Ontology Analysis and Enrichment Map

Proteomic results were ranked according based on fold change and submitted to a GSEA Preranked analysis in GSEA (v. 4.0.2) with 1000 permutations. Terms smaller than 15 genes or bigger than 500 were discarded as previously reported. The enrichment map was generated in Cytoscape (3.7.1) using Enrichment map plugin (3.2.0) using the following thresholds: p value <0.05, FDR <0.001. Alternatively, proteomic results were submitted to ontology analysis using Enrichr. Node size indicates the number of proteins per node. Major clusters are circled, and the associated name represent the major functional association.

### KFERQ-Like Motif Enrichment

Analysis of content in KFERQ-like motif was performed using the publicly available tool: https://http://tinyurl.com/kferq.

### Calculation of CMA Activation Score

Data set used were from (Grubman et al., Nat. Neurosci. (2019) 22: 2087-2097; Mathys et al., Nature (2019) 570: 332-337). Raw counts per cell and metadata (cell type identification and patients' information) were obtained from the Synapse portal (https://www.synapse.org/#!Synapse:syn18485175). Filtered counts provided by (Mathys et al., 2019) were used. Single cell gene expression data and metadata were obtained from http://adsn.ddnetbio.com/ for the dataset (Grubman et al., 2019) Prior to calculations, counts were log normalized using SCANPY (v.1.4.5) (Wolf et al., 2018). For each cell, a CMA activation score was calculated. To do so, each element of the CMA network was attributed a weight. As LAMP-2A is the rate limiting component of CMA, it was given a weight of 2. Every other element received a weight of 1. Then, every element was attributed direction score that is +1 or −1 based on the known effect of a given element on CMA activity. The score was then calculated as the weighted/directed average of expression counts of every element of the CMA network. To ease understanding of transcriptional CMA activity changes between conditions, the CMA score was expressed as fold of healthy individuals within a given cell type.

### Quantification and Statistical Analysis

All data presented are mean±s.e.m. with *: p<0.05, **: p<0.005, **: p<0.0005. Prior to statistical testing, normality was assessed using the Shapiro Wilk test. Parameters with two groups were compared using unpaired, two-tailed t-test or Mann Whitney U test. Parameters with more than two groups were compared using one-way ANOVA and Tukey's post-hoc analysis, Kruskal-Wallis test followed by Dunn's post-hoc analysis or two-way ANOVA followed by Sidak post-hoc analysis. The number of animals used per experiment was calculated through power analysis based in previous results. Statistical analyses were performed either in GraphPad Prism 8.0 or using Python (Python software foundation v.3.7.4 available at https://www.python.org/) and the scientific python stack: scipy (v.1.3.1), numpy (v.1.17.2) (van der Walt et al., 2011), and matplotlib (v.3.1.1).

### Materials and Methods for the Chemical Synthesis of CMA Activators

All chemical reagents and solvents were obtained from commercial sources (Aldrich, Acros, Fisher) and used without further purification unless otherwise noted. Chromatography was performed on a Teledyne ISCO CombiFlash Rf 200i using disposable silica cartridges (4, 12, and 24 g). Analytical thin layer chromatography (TLC) was performed on aluminum-backed Silicycle silica gel plates (250 μm film thickness, indicator F254). Compounds were visualized using a dual wavelength (254 and 365 nm) UV lamp, and/or staining with CAM (cerium ammonium molybdate) or KMnO₄ stains. NMR spectra were recorded on Bruker DRX 300 and DRX 600 spectrometers. ¹H and ¹³C chemical shifts (d) are reported relative to tetramethyl silane (TMS, 0.00/0.00 ppm) as internal standard or to residual solvent (CDCl₃: 7.26/77.16 ppm; dmso-d6: 2.50/39.52 ppm). Mass spectra (ESI-MS) were recorded on a Shimadzu LCMS 2010EV (direct injection unless otherwise noted). High resolution mass spectra (HRMS) were recorded on an Orbitrap Velos high resolution mass spectrometer at the Proteomics Facility of Albert Einstein College of Medicine.

The following compounds, which have been previously disclosed, are used in the examples.

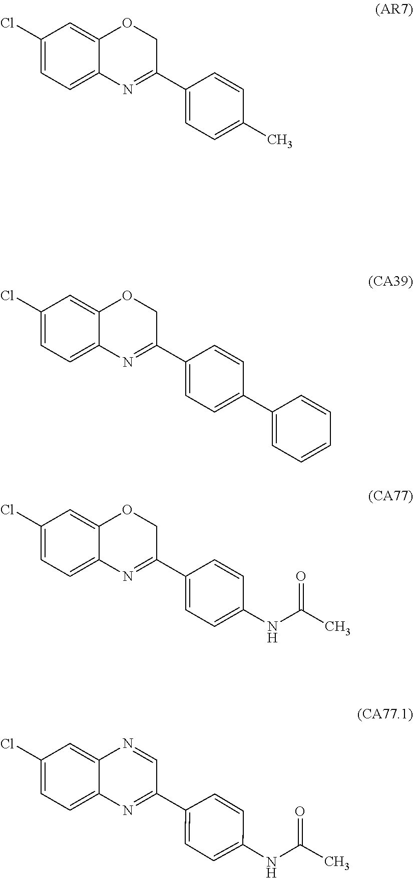 [SMILES: Cc1ccc(C2=Nc3ccc(Cl)cc3OC2)cc1 ; Clc1ccc2c(c1)OCC(c1ccc(-c3ccccc3)cc1)=N2 ; CC(=O)Nc1ccc(C2=Nc3ccc(Cl)cc3OC2)cc1 ; CC(=O)Nc1ccc(-c2cnc3cc(Cl)ccc3n2)cc1]

### Example 1. Neuronal-Specific CMA Deficiency Lead to Behavioral Impairments

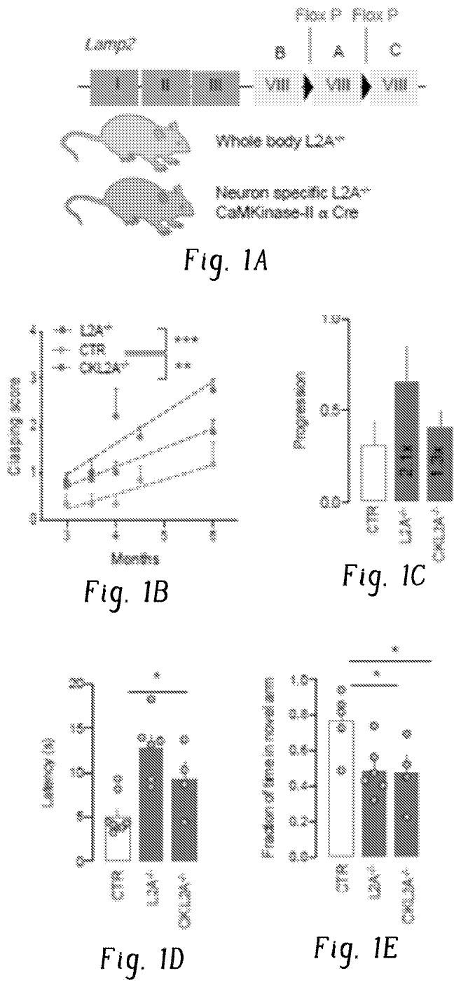

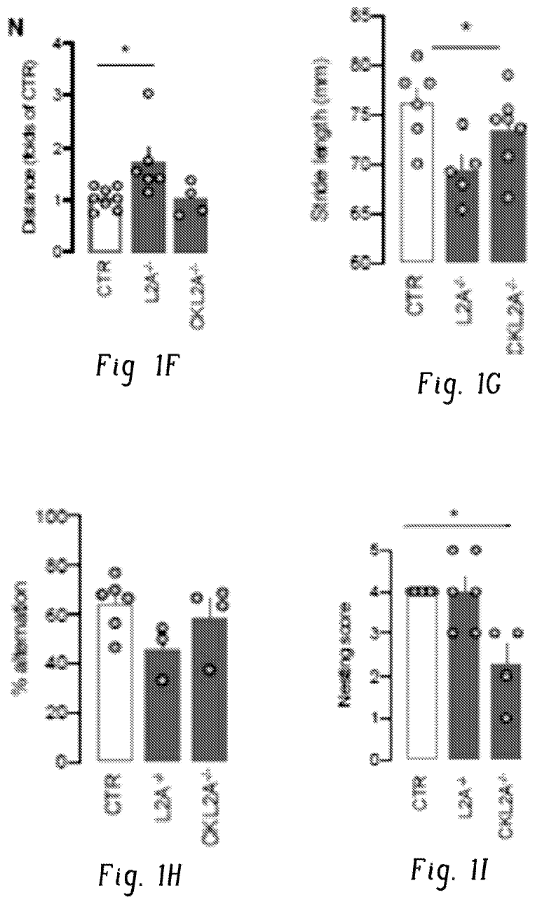

We generated a neuronal-specific CMA-deficient mouse model (CKL2A^{-/-} mice) by removing L2A in excitatory neurons (expressing calcium/calmodulin-dependent protein kinase II (α-CaMKIIα)). We breed L2A^{flox/flox} mice with a CaMKIIα-driven Cre recombinase transgenic mouse line for selective deletion of the Lamp2 gene exon encoding for the cytosolic and transmembrane domain of the L2A protein. (FIG. 1A). As comparison, we used mice knockout for L2A in the whole body (hereafter named L2A^{-/-}). We confirmed the selective deletion of L2A and not other Lamp2 isoforms in both mouse models; the remaining L2A signal in CKL2A^{-/-} mice originates from other brain cell types). Both mouse models were born at Mendelian frequency, proportional male/female ratios and only L2A^{-/-} showed an initial delay on body weight gain that normalized by 6 months. The transgenic mice displayed a discrete reduction in life expectancy that was more noticeable for CKL2A^{-/-} mice.

Behavioral testing revealed higher scores and faster hindlimb clasping progression, a phenotype of neurological disorder models, in L2A^{-/-} and CKL2A^{-/-} mice than control (CTR) littermates as early as 3 months of age (FIGS. B,C). Both mouse models showed higher latency in the negative geotaxis test, indicative of vestibular/sensorimotor dysfunction (FIG. 1D) and reduced short-term memory in a Y-maze (FIG. 1E). Interestingly, only L2A^{-/-} mice displayed impaired locomotion, evidenced by (i) hyperlocomotion in the open-field, reminiscent of some AD models (Min et al., 2015; Takeuchi et al., 2011) (FIG. 1F) and (ii) reduction in stride length (FIG. 1G), a feature of PD gait (Fernagut et al., 2002). In addition, CKL2A^{-/-} mice displayed a trend towards reduced spatial working memory (FIG. 1H) and significant reduction in nest building (FIG. 1I), a common phenotype of neurodegeneration in mice (Hernandez et al., 2019). Overall, loss of CMA in only excitatory neurons reproduced most of the behavioral outputs observed upon systemic loss of CMA.

### Example 2. Neuronal CMA Deficiency Leads to Proteostasis Collapse

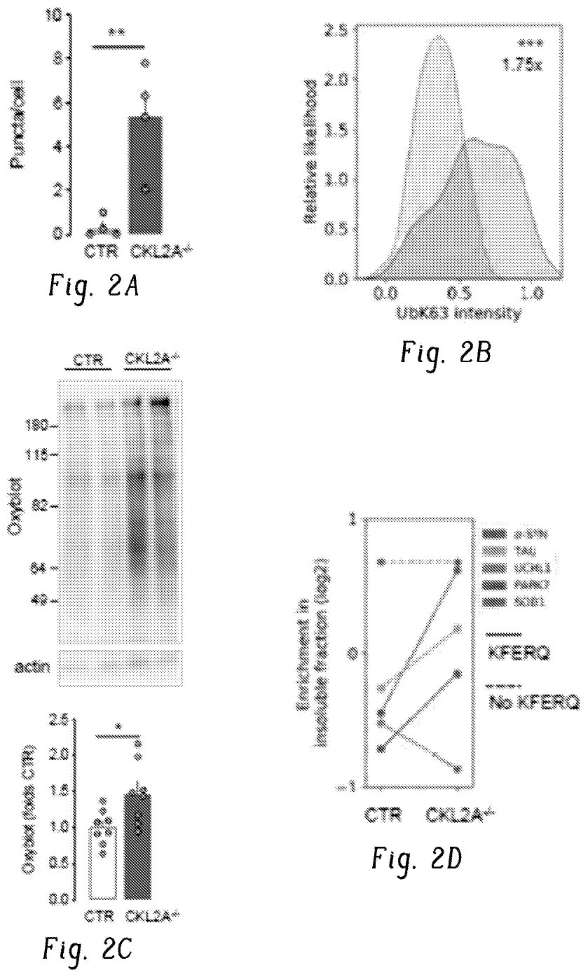

By 6 months of age, both mice with systemic or neuronal-specific CMA loss showed thicknesses of CA1, dentate gyrus and cortex as well as hippocampal surface undistinguishable from their respective CTR littermates (FIG. 2A). Number of astrocytes and microglia was unchanged in the hippocampus of L2A^{-/-} and CKL2A^{-/-} mice, and only a small increase of astrocyte area was noticeable in CKL2A^{-/-} mice.

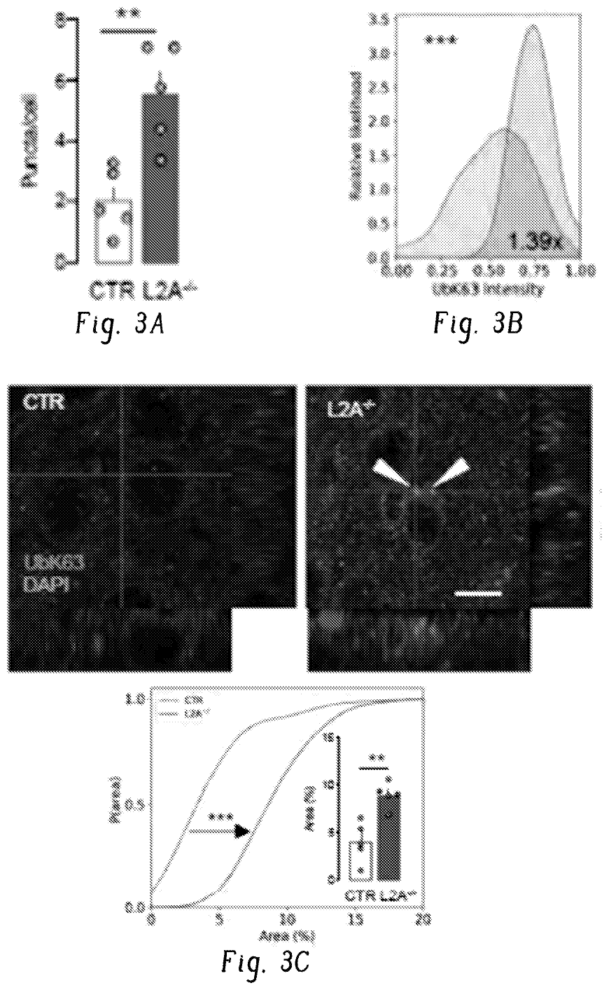

Analysis of overall cellular proteostasis revealed lipofuscin deposits (cross-linked oxidized proteins and lipids) (FIG. 3A) and K63-linked ubiquitinated proteins inclusions—usually targets of lysosomal degradation (Kraft et al., 2010) (FIGS. 3B, 3C) in the hippocampus of L2A^{-/-} mice at 6 months of age. Similar features were noticeable at this early age in excitatory pyramidal neurons in the hippocampus of CKL2A^{-/-} mice, but not in regions occupied mostly by interneurons (i.e the stratum radiatum) or in glial cells. Immunoblot analysis of CKL2A^{-/-} mice cortex revealed accumulation of oxidized proteins (FIG. 2C), hydroxynonenal protein adducts and of ubiquitinated proteins, specifically with K63-linkage (FIG. 1H), further supporting that loss of CMA results in profound disruption of neuronal proteostasis.

To analyze the extent of proteome compromise upon neuronal CMA blockage, we isolated the sarkosyl-insoluble fraction from the cortex of CTR and CKL2A^{-/-} mice and performed comparative quantitative proteomics. We found a significant enrichment of proteins in the insoluble fraction of CKL2A^{-/-} mice brains. Most insoluble proteins (76%) contained KFERQ-like motifs, mainly those that do not require posttranslational modifications for hsc70 recognition (˜52% in CKL2A^{-/-} sarkosyl-insoluble fraction vs. ˜47% in the whole murine proteome (Kirchner et al., 2019)). Prone-to-aggregate proteins bearing KFERQ-like motifs such as α-syn, tau, UCHL1 and PARK7, displayed a shift towards insolubility in CKL2A^{-/-} brains, whereas this was not the case for SOD1 that lacks the motif (FIG. 2D). The only exception was UCHL1 (FIG. 2D) which, interestingly, bears a phosphorylation-generated motif. To investigate the status of the supersaturated proteome—the one at-risk of aggregation due to protein concentrations close to their solubility limit (Ciryam et al., 2013; Kundra et al., 2017)—we analyzed the supersaturation score (expression of a protein relative to its solubility limit) and found a remarkable increase (20.37 folds) of the supersaturation score σ_{f} of proteins enriched in the insoluble fraction of CKL2A^{-/-} mice compared to the whole proteome. These findings support that the proteome that shift to insolubility upon CMA loss is part of the supersaturated proteome. In fact, proteins bearing constitutive or acetylation-generated KFERQ-like motifs showed higher σᵤ and σ_{f} supersaturation scores, indicative of their tendency to form aggregates from unfolded or folded state, respectively.

Taken together, these results demonstrate a profound collapse of the proteome upon neuronal CMA blockage.

### Example 3. Neuronal CMA Deficiency Leads to Functional Dysregulation

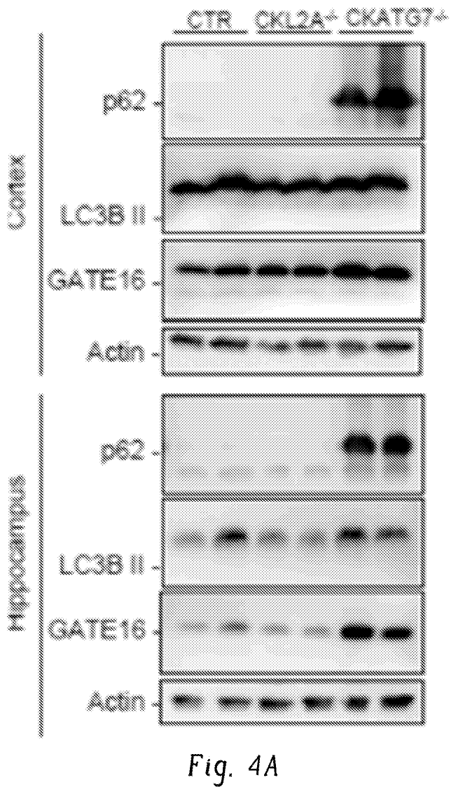

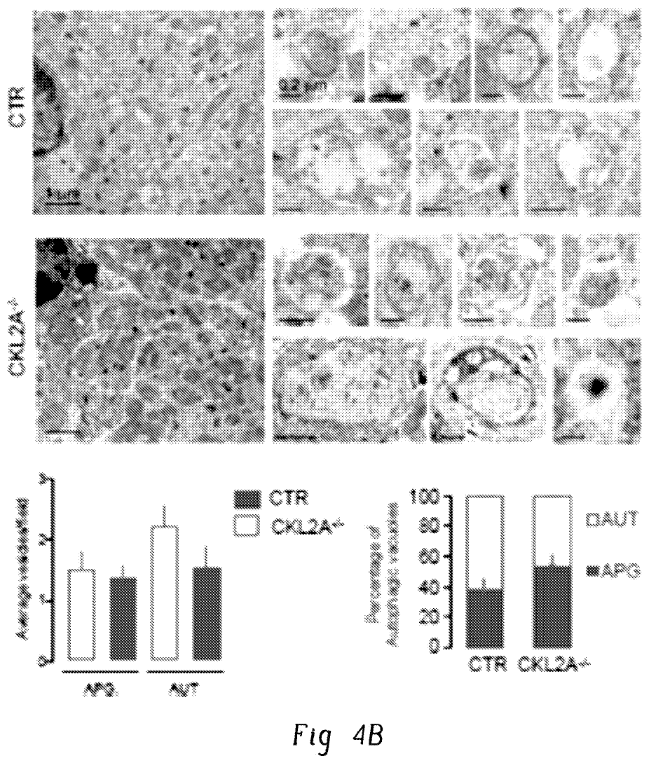

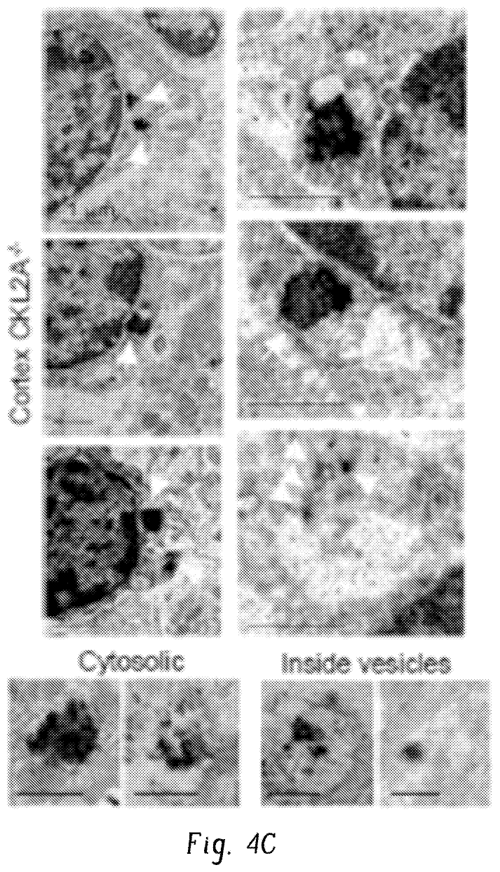

Neuronal protein aggregates have been described upon blockage of macroautophagy. However, the loss of proteostasis in CMA-deficient brains was not due to macroautophagy malfunction. Contrary to the macroautophagy failure observed when all three spliced variants of LAMP2 were eliminated, steady-state levels of autophagosome components (LC3 and GATE16), macroautophagy adaptors (p62) and lysosomal membrane proteins (LAMP1) were comparable in cortex and hippocampus of L2A^{-/-}, CKL2A^{-/-} and their corresponding CTR littermates (FIG. 4A; note the marked p62 accumulation in macroautophagy-defective mice in the same neurons (CKATG7^{-/-} mice)). Ultrastructural analysis revealed that number and state of maturation of autophagic compartments was indistinguishable between CKL2A^{-/-} and CTR mice (FIG. 4B), while perinuclear electrodense material (likely protein inclusions) was only observed in CKL2A^{-/-} neurons (FIG. 4C).

We used comparative proteomics of the insoluble proteome of CKL2A^{-/-} and CKATG7^{-/-} mice to elucidate the specific contribution of CMA and macroautophagy to neuronal proteostasis (FIG. 5A; large functional multiprotein complexes present in the sarkosyl-insoluble fraction of CTR mice were eliminated from the transgenic mice analysis). About half of the proteins in the sarkosyl-insoluble fractions were the same, independently of the autophagic pathway blocked, whereas the rest were distinctive for CKL2A^{-/-} or CKATG7^{-/-} brains (FIG. 5B). Rank-rank hypergeometric overlap (RRHO) analysis (Plaisier et al., 2010) showed that the most enriched proteins were the less similar. We propose that proteins present in both CKL2A^{-/-} and CKATG7^{-/-} aggregates are metastable proteins trapped in the insoluble fraction independently of the cause of proteostasis failure, whereas proteins specific for the autophagic pathway blocked reflect the sub-proteome selectively regulated by each of them.

Gene-set enrichment and Enrichment Map analyses of proteins in the insoluble fractions of only CKL2A^{-/-} or CKATG7^{-/-} mice brains (FIG. 5C) revealed that clusters associated with macroautophagy deficiency were related with cell cycle and with ubiquitin-proteasome catabolic processes (FIG. 5C) while those associated with CMA deficiency were related with protein trafficking, cation (mostly calcium) homeostasis and metabolism (FIG. 5C).

We experimentally confirmed that the presence of these proteins in the insoluble fraction had functional consequences in the CMA-defective neurons. Measurement of the extracellular acidification rate (ECAR) revealed pronounced decrease of glycolysis in CKL2A^{-/-} neurons (FIG. 5D). Although macroautophagy blockage has been shown to also affect neuronal glycolysis, side-by-side comparison demonstrated that L2A and ATG7 knockdown affected glycolysis properties differently. Most glycolytic enzymes, confirmed as bona fide CMA substrates in other cell types and tissues, also increased in the insoluble fraction of CKL2A^{-/-} mice, with pyruvate dehydrogenase (PDH)—that catalyzes formation of acetyl-coenzyme A (acetyl-CoA) from pyruvate—showing the most dramatic increase. Reduced acetyl-CoA levels due to PDH entrapment in aggregates, could explain the alterations in the acetylome of CMA-defective neurons detected by mass-spectrometry. The brain proteome of CKL2A^{-/-} mice contains lower number of unique acetylation sites (−11%) and of acetylation events per site (−30%) compared to CTR littermates. As described in other conditions with limited acetyl-CoA availability such as aging (Pietrocola et al., 2015), we also found that a subset (30%) of the CKL2A^{-/-} proteome was hyperacetylated. The hypoacetylated proteome was functionally associated with glycolysis-related terms and the hyperacetylated with ion transport.

To further confirm the functional consequences of the shift toward insolubility of the CMA-deficient neuronal proteome, we analyzed a second protein cluster—“protein targeting and localization”—enriched in proteins related to endocytosis and to actin remodeling (FIG. 5C). We found significant accumulation of clathrin and marked reduction of the clathrin adaptor protein AP2α in L2A^{-/-} mice brains (FIG. 5E-5F), that are likely responsible for the significant decrease in endocytosis observed in L2A-defficient cells (FIG. 5G). Among the actin remodeling proteins, L2A^{-/-} mice accumulate Arpc2, an essential regulator of actin polymerization (FIG. 5H), which could be responsible for the actin cytoskeleton changes observed in the hippocampus of L2A^{-/-} mice (FIG. 5I), including the formation of actin-rich rods, reminiscent of Hirano bodies described in AD and other tauopathies,

These results demonstrate that defective neuronal function upon CMA blockage is a combination of a direct impact on the solubility of the neuronal proteome and indirect effects due to the functional disruption of aggregate-entrapped CMA substrate proteins.

### Example 4. CMA is Inhibited in Early Alzheimer's Disease Stages

The alterations in proteostasis and neuronal function upon direct neuronal CMA blockage, indicate that reduced CMA activity increases vulnerability and accelerates disease progression in neurodegenerative disorders.

To test this possibility, we measured CMA in a mouse model of tau-mediated proteotoxicity expressing mutant human tau (hTau^{P301L}) under the Thy 1.2 promotor (line pR5). We crossed hTau^{P301L} mice with a transgenic mouse model that expresses systemically a KFERQ-tagged fluorescent protein Dendra2 (KFERQ-Dendra), recently developed by our group. This reporter protein, when targeted for degradation via CMA, highlights lysosomes as fluorescent puncta, that can be counted as read out of CMA activity. Hippocampal neurons of KFERQ-Dendra-hTau^{P301L} mice displayed a significant reduction in the number of fluorescent puncta compared to control KFERQ-Dendra mice (FIGS. 6A). CMA inhibition, at least at this stage of the pathology, preferentially occurred in neurons, while astrocytes showed no difference in number of KFERQ-Dendra⁺ puncta per cell (FIG. 6B).

Insights on the CMA status of the neurodegenerating human brain have been so far limited to analysis of L2A expression in PD blood cells or whole brain extracts. We took advantage of a recently published single-nucleus (sn) RNAseq dataset from prefrontal cortex of 24 healthy individuals and 24 AD patients grouped according to their Braak score in 3 categories: low (Braak 0, I, II), medium (Braak III, IV) and high (Braak V, VI) (Mathys et al., Nature (2019) 570: 332-337, and extracted the expression level of every element in the CMA network (FIG. 7E). Although mRNA and protein levels correlate weakly in single bases (Vogel and Marcotte, 2012), considering a group of genes, as a functional network, is a robust approach to infer functional outputs (Freer et al., 2016). Gene expression analysis revealed transcriptional inhibition of almost the entire CMA network in excitatory neurons, whereas in inhibitory neurons, we observed increased expression of two CMA negative regulatory elements, AKT1 and Retinoic-acid receptor α (RARα) (FIG. 6E). In comparison, we only detected modest changes in expression levels of the transcription factor EB (TFEB), a master regulator of macroautophagy and lysosomal biogenesis.

To infer the impact of the transcriptional differences on CMA output and compare CMA activity among cell types and pathological stages, we next defined a CMA activation score. This score is a weighted average of the expression level of every element of the CMA network. Higher scores could result from (i) increased expression of effectors or positive modulators or (ii) decreased expression of negative modulators, whereas changes in opposite direction will render lower CMA activation scores. We experimentally validated this score in cultured cells exposed to pro-oxidant conditions (which activate CMA) or to a chemical CMA activator.

Using the CMA activation score in the snRNAseq dataset from the control and AD human brains, we found an inhibition of the CMA network already at early pathology stages in both excitatory and inhibitory neurons, followed by deeper inhibition at later disease stages (FIG. 6D). In agreement with our observations in KFERQ-Dendra-hTau^{P301L} mice (FIG. 6B), we observed no changes in astrocytes and microglia and an increase in CMA score in oligodendrocytes, mostly driven by higher expression of CMA effectors and positive modulators (FIG. 6C).

The CMA activation score of excitatory neurons revealed significant negative correlation with different quantitative pathology markers such as the Braak stage (FIG. 3C) and the NIA-Reagan score (that combines neurofibrillary tangles and neuritic plaques). Inhibitory neurons, despite reduced CMA at late Braak stages, did not show correlation with these neuropathology markers . The CMA activation index was reduced in most of the 13 subclusters of excitatory neurons identified in this dataset (Mathys et al., 2019), although in some of them CMA inhibition was more gradual (Ex4 neurons) or only at late stage (Ex9 population), and in others there was no inhibition (Ex8 population) or even CMA activation (Ex6 population), supporting the recently described diversity within neuronal population (Fan et al., 2018), and neuronal-type specific differences in vulnerability to CMA loss in AD. When considering the distribution of these neuron subpopulations into cortical layers (Mathys et al., 2019), layer II-III excitatory neurons displayed the highest inhibition of CMA early in the disease. These results suggest spatial-temporal decrease in CMA activity in the cortex coincident with the development of pathology, especially in excitatory neurons, known to be highly vulnerable in tauopathies (Fu et al., 2019).

Altogether, these findings reveal a previously unknown neuron-specific transcriptional downregulation of CMA early in AD, that could add to the direct toxic effect of pathogenic AD proteins on CMA, as reported before (Caballero et al., 2018).

### Example 5. CMA Deficiency Accelerates Pathology in a Mouse Model of AD

To evaluate the contribution to disease progression of the observed early inhibition of CMA in AD, we imposed CMA loss to a mouse model of AD-related proteotoxicity by breeding triple transgenic (Tg) AD mice (expressing APP^{Swe}, PS2^{N141I} and hTau^{P301L}) with L2A^{-/-} mice (hereafter named Tg-L2A^{-/-}) (FIG. 9A). At 8 months, when Tg mice displayed modest Aβ pathology, Tg-L2A^{-/-} mice already showed profound Aβ deposition and accumulation of S422 phosphorylated Tau (pS422-tau) (FIG. 8A). Immunoblot analyses revealed that, despite comparable levels of murine, human tau (FIG. 8B-8D) and K63-linked ubiquitinated proteins (FIG. 7B), Tg-L2A^{-/-} mice displayed significantly higher accumulation of phosphorylated tau (FIG. 8B, 8E) and of aggregated tau (HT7 epitope) and pS422-tau in the sarkosyl-insoluble fraction (FIG. 10J). CMA loss did not increased accumulation of full-length APP in Tg mice, but significantly increased levels of APP C-terminal fragments (CTFs) and of Aβ42 peptide (FIGS. 8B, 8F-8I).

Total tau levels displayed similar progression in Tg and Tg-L2A^{-/-} mice brains at 5, 8 and 12 months of age, while pS202/T205 tau levels were constantly higher in Tg-L2A^{-/-} mice and both, pS422 and aggregated tau showed higher levels and faster accumulation rates (2.5 to 3.1 folds) (FIG. 8K). These findings suggest that reduced CMA activity, observed early in AD patients and further accentuated with aging, could aggravate brain proteotoxicity and accelerate pathology progression.

To identify how CMA deficiency aggravates AD, we used comparative quantitative proteomic of brain cortex from CTR, Tg, L2A^{-/-}, and Tg-L2A^{-/-} mice. We found 152 differentially expressed proteins between CTR and L2A^{-/-} mice (FIG. 7C) and only 5 differentially expressed proteins between CTR and Tg mice (7D). Interestingly, Tg-L2A^{-/-} mice showed higher number of differentially expressed proteins than Tg and L2A^{-/-} mice combined (FIG. 9A and 7E). A hierarchical clustering analysis grouped L2A^{-/-} and Tg-L2A^{-/-} mice together (FIG. 9A) and proteome-wide transition mapping approach (Stein et al., 2014) revealed strong overlap between L2A^{-/-} and Tg-L2A^{-/-} signatures, both supporting dominance of the CMA deficiency phenotype in Tg-L2A^{-/-} mice.

Imposing CMA blockage in the context of AD-related brain proteotoxicity resulted in quantitative and qualitative proteome changes not recapitulated by any of the genotypes separately. Direct comparison of Tg-L2A^{-/-} and L2A^{-/-} proteomes and Tg-L2A^{-/-} and Tg proteomes revealed little overlap in differentially expressed proteins. Gene ontology and network analysis to identify biological processes altered only in Tg-L2A^{-/-} mice (FIG. 9B) revealed clusters related with cellular metabolism (including calcium homeostasis), lipid metabolism and cellular organization (mostly of actin cytoskeleton), which suggest that metabolic and architectural dysfunctions of CKL2A^{-/-} mice could be further altered in the context of AD pathology. Additional Tg-L2A^{-/-} selective changes included proteins associated with lipoprotein catabolism, ubiquitin/proteasome, and regulation of amyloid processing, which contained APP and the well-known AD risk factor, apolipoprotein-E.

We reasoned that our mouse model of AD with defective CMA, may recapitulate, at least in part, human disease progression, since CMA activity will further decrease in AD patients as they age. Comparison of the proteomic changes in brains of low and high pathology controls, asymptomatic and symptomatic AD patients from the Consensus study (Johnson et al., Nature Medicine (2020) 26: 769-780) with those in our Tg-L2A^{-/-} mice, supports this possibility as we found: (i) the highest positive correlation between proteome changes in AD patients and Tg-L2A^{-/-} mice brains, (ii) stronger overlap between AD patients and Tg-L2A^{-/-} mice signatures from proteome-wide transition mapping than with Tg or L2A^{-/-} mice (FIG. 9C) and (iii) higher similaity in changes in levels of regulatory proteins of amyloid fibril between AD patients and Tg-L2A^{-/-} mice (FIG. 9D).

Reduced CMA may also accelerate the underlying autophagy/lysosomal found in AD brains. Contrary to Tg and L2A^{-/-} mice, Tg-L2A^{-/-} mice showed accumulation of p62 and GATE-16 (suggestive of reduced autophagic flux), marked increase in lysosomal hexosaminidase activity and accumulation and mislocalization of cathepsin D, as occurs in the AD brain (note that cathepsin D levels increase in homogenate but not in lysosomes). As expected, elimination of L2A alone or in the Tg background did not have major effect in levels of other CMA components.

Overall, these results support the aggravating effect of CMA loss with age on neurodegenerative disease progression due to synergistic, and not merely additive, alterations of the brain proteome.

### Example 6. Chemical Activation of CMA Improves Pathology in Complementary AD Mouse Models

To test if CMA activation could be protective against AD-related neuroproteotoxicity, we performed extensive medicinal chemistry on AR7, one of the generated CMA activators, to make derivatives suitable for in vivo administration (see Methods section). The derivative used in this study (CA77.1, thereafter CA) activates CMA in vitro in dose- and time-dependent manner without affecting macroautophagy (FIGS. 10A-10D), and when administered in vivo demonstrated brain penetrance with favorable pharmacokinetics (FIGS. 10E-10F), activation of CMA in brain of KFERQ-Dendra mice (Dong et al., 2020), and absence of blood or major organs toxicity after 3 months of daily treatment (FIGS. 10G-10J).

We first administered CA to PS19 mice, which express tau with the frontotemporal dementia mutation P301S. We used a clinically relevant administration design (FIG. 11A) with oral daily doses of CA (30 mg/kg body weight) for 6 months starting at 3 months of age, when PS19 mice exhibit synaptic loss and tau seeding activity, but before overt tau pathology. CA administration normalized the previously described locomotor hyperactivity of PS19 mice to control levels (FIGS. 11B, 11C) and reduced the levels and number of neurons containing pathogenic tau conformations (recognized by the MC1 antibody) in the hippocampus, amygdala and piriform cortex (FIGS. 11D-11H). A semi-quantitative analysis of hyperphosphorylated tau (S202/T205—AT8 antibody) also revealed less aggressive tau pathology in the hippocampus of CA-treated mice, with tau Type III pathology observed in 60% of vehicle and 10% of CA-treated PS19 mice (FIG. 10K). Immunoblot analyses confirmed that CA-treated mice showed lower levels of conformationally aberrant, S422 and AT8 phosphorylated, oligomeric and insoluble forms of tau (FIGS. 11F-11I and FIGS. 10L-10N). CA did not reduce total levels of human tau, indicating improved processing rather than reduction of expressed levels (FIG. 11M). The higher number of microglial cells and presence of large Iba1-positive cells with rod-like dystrophic morphology in vehicle-treated PS19 mice were reduced upon CA treatment (FIGS. 11K-11M and FIGS. 10O, 10P).

Next, we used the triple transgenic mouse model of AD (Tg) (Grueninger et al., Neurobiol. Dis. (2010) 37: 294-306) to explore the effect of pharmacological activation of CMA on combined tau and Aβ proteotoxicity. Tg mice were given daily oral doses of CA (30 mg/kg body weight) for 4 months starting at 8 months of age (after symptoms' onset and when b-amyloid plaques are already detectable. CA-treated Tg mice had better visual memory, decreased anxiety- and depression-like behaviors, slower clasping progression and increased performance in horizontal grid test than those receiving the vehicle (FIGS. 12A-12F). Histopathology revealed significantly reduced β-amyloid and tau-related pathologies in hippocampus and cortex of CA-treated mice evidenced as: reduced area and size of immature (MOAB2-positive) and mature (6E10 and thioflavin S-positive) amyloid plaques (FIGS. 12G-12L and FIG. 13A-13C), lower number of deposits depending on the deposit maturity (FIG. 12M), and reduced levels of the early T231 phosphorylated Tau (FIGS. 12J, 12 and FIGS. 13D, 13E), especially in 6E10-positive areas, in support of lower number of neurons bearing pathology (FIG. 12O). Immunoblot analysis demonstrated significant reduction of the mature phosphosite AT8 in CA-treated Tg mice (FIG. 13F). We also found lower number of microglia and astrocytes in the dorsal hippocampus and less association of glial cells with amyloid plaque-like deposit (FIGS. 13G-13I), revealing reduced gliosis in the CA-treated group.

These results indicate that pharmacological CMA activation using a clinically relevant design has a beneficial effect on AD-related pathology.

### Example 7. CMA Upregulation Protects Against Atherosclerosis in Mice

To experimentally test the proposed protective effect of CMA activation—observed early in the disease in mice and in human plaques—and to evaluate the possible therapeutic value of CMA modulation in atherosclerosis, we directly upregulated CMA activity in mice exposed to a proatherosclerotic challenge. To that end, we used an inducible transgenic mouse model (hL2AOE), expressing the human form of LAMP-2A, which we induced after the observed drop in LAMP-2A levels in early plaques (FIG. 14A). We confirmed that weight and circulating cholesterol were indistinguishable between WT and hL2AOE mice before the treatment. After the pro-atherosclerotic challenge, although circulating cholesterol and triglyceride levels were only discretely reduced, increasing CMA activity markedly decreased the fraction of both lipids in the VLDL and LDL fractions (FIG. 19, A, B). As anticipated, the improved lipid profile of the hL2AOE group associated with an increase in insulin sensitivity. The hL2AOE mice also showed a trend towards ameliorated profile of multiple pro-inflammatory cytokines (i.e., PAI1, CCL3, 4 and G-CSF) that we found modified in the opposite direction in CMA-defective mice upon the same challenge. Analysis of the atherosclerotic plaque revealed that hL2AOE mice exhibited smaller lesions with reduced necrotic cores and less calcification (FIG. 19, C-E) although collagen content, plaque stage and number of VSMC and macrophages were comparable in both groups of mice. Principal component analysis with 12 variable data shows that Ctrl and hL2AOE mice groups are distributed in different regions in support of these groups evolving differently upon the lipid challenge (FIG. 19, F, G). Overall, these findings support both systemic and vascular beneficial effects of CMA upregulation and highlight the therapeutic potential of activating CMA to prevent atherosclerotic disease progression.

### Example 8. Human Carotid CMA Response to Pro-Atherosclerotic Conditions

Our in vitro and in vivo findings support that CMA upregulation may be part of the vascular response to pro-atherosclerotic challenges. To test whether that was also the case in human atherosclerosis, we first confirmed the presence of the CMA receptor in plaque VSMC and macrophages using costaining for LAMP-2A and αSMA/CD68. Analysis of levels of LAMP-2A in human atherosclerotic plaques from asymptomatic patients at different plaque stages revealed that LAMP-2A levels at the plaque increase gradually with disease progression (graded as plaques with moderate intimal thickening (IT), pathological intimal thickening (PIT), thick fibrous cap atheroma (TkFCA) and plaques with intraplaque hemorrhage (IPH)). The increase in LAMP-2A protein levels originates mainly from LAMP-2A mRNA upregulation. In fact, LAMP-2A mRNA levels directly correlated with the size of the plaque but not the necrotic core. To determine the cell type mainly contributing to the elevated levels of LAMP-2A at the plaque, we analyzed the correlation between LAMP-2A levels and different cell types and found a direct correlation between LAMP-2A and CD68, a marker of macrophages and foam cells, in human atherosclerotic plaques. We interpreted these changes in LAMP-2A levels as an attempt of the plaque cells, mostly macrophages, to upregulate this autophagic pathway in response to the pro-atherosclerotic changes prior to clinical events, as we observed in the experimental mouse model. In fact, holistic analysis of the CMA transcriptional network using single cell RNA seq (scRNAseq) from human atherosclerotic plaques (from (8)) confirmed macrophages as the cells with the highest expression of CMA effectors (LAMP-2A and (HSC70) when compared to endothelial and smooth muscle cells from the same plaques. To evaluate possible changes of CMA after the clinical event, we performed immunoblot for LAMP2 in carotid segments from patients who suffered one or two clinical vascular events.

This revealed a significant decrease in LAMP2 levels in carotid segments from all patients who develop a second event (FIG. 18A). This decrease in LAMP2, seems to be driven mostly by the female patients in this group, that were the ones displaying the most pronounced changes in overall LAMP2 content (FIG. 18B; changes cannot be attributed to differences in patient's ages that were 66.1+3.4 and 70.4+3.8 for first and second event, respectively in female patients and 65.6+1.3 and 74.3+1.2 for the same groups in male patients). The observed changes, seem selective for LAMP2, rather than an overall reduction in the lysosomal content since levels of cathepsin D, another lysosomal marker, remained unchanged (FIG. 18A,B). We have recently developed and validated an algorithm that allows inferring CMA activity based on the weighted averaged expression of the genes known to contribute as effectors or regulators to CMA. Analysis of the CMA score using transcriptomics from patient plaques predicted reduction of CMA activity in unstable atherosclerotic plaques when compared with stable ones (FIG. 18C). Deconvolution analysis highlighted an overall lower expression of most network elements, especially of positive lysosomal modulators, despite an increase of effectors (LAMP-2A and HSC70). This increase in LAMP9 2A expression can be interpreted as an attempt to compensate for the pronounced decrease in LAMP2A protein levels in these patients, likely due to reduced stability of this protein as previously observed upon persistent lipid challenges.

Our findings suggest that reduced levels of LAMP-2A, and lower CMA activity could be a predictor of the risk of suffering a second clinical event.

### Example 9. CMA Blockage Exacerbates Atherosclerosis in a Murine Experimental Model

We used the recently developed transgenic mouse model expressing a fluorescence reporter for CMA (KFERQ-PS-Dendra2 mice) that allows measuring CMA activity in vivo to determine the status of CMA in the vasculature and its possible changes during atherosclerotic plaque development. When KFERQ-PS-Dendra CMA substrate is delivered to lysosomes, CMA activity is detected as fluorescent puncta against the diffuse fluorescent cytosolic pattern. Using aortas from healthy mice and two-photon microscopy in fixed tissue or intravital two photon microscopy, we found fluorescent puncta in cells in the media (VSMC) and to less extent in the intima (endothelial cells). Injection of fluorescent dextran that highlights the lysosomal compartment upon internalization from the bloodstream by endocytosis demonstrated colocalization with the Dendra signal in a fraction of lysosomes, in support of active CMA in the vasculature under basal conditions. When we promoted atherosclerosis development in KFERQ-PS-Dendra2 mice through hypercholesterolemia (using injection of adeno-associated virus 8-mediated overexpression of proprotein convertase subtilisin/kexin type 9 (AAV8-PCSK9) and a high cholesterol-containing diet (Western type diet, WD) for 12 weeks), aortas from these mice revealed a marked reduction in the number of fluorescent puncta that was almost absent in the plaque (FIG. 14A) that shows maximal projections throughout the plaque area. Co-staining of these atherosclerotic aortas with the VSMC marker α-smooth muscle actin (α-sma) and the macrophage marker cluster of differentiation 68 (CD68), revealed almost no CMA activity in either cell type. Immunohistochemistry of the plaque demonstrated that levels of LAMP-2A, the limiting component for CMA, can be detected both in VSMC and macrophages by 6 weeks of plaque formation, but LAMP-2A levels significantly decreased in more advanced stages (12 weeks) of murine atherosclerotic disease in both VSMC and macrophages, in agreement with the observed reduction in CMA activity (FIG. 14B).

To determine if the initial upregulation of LAMP-2A in response to the dietary challenge was protective and whether reduced CMA contributes to disease progression, we used a mouse model with systemic blockage of CMA (constitutive knock-out for LAMP-2A, L2AKO. At 3 months of age, L2AKO mice on chow diet display slightly lower body weight and circulating total cholesterol levels than wild-type (WT) littermates. When L2AKO mice were fed WD for 12 weeks, we observed a marked increase in total circulating cholesterol and triglyceride (TG) levels (FIG. 14C,D), mainly in the very-low-density lipoprotein (VLDL) and LDL fractions. Atherosclerotic plaques in the aortic root of L2AKO mice were larger than in WT mice (approx. 39%) (FIG. 14E), with a noticeable trend toward bigger necrotic cores, lower cellularity, and significantly more advanced plaques (FIG. 14F). Plaques in the CMA-incompetent mice also had more collagen content, thicker fibrous cap, yet higher abundance of calcifications (FIG. 14G). In addition, the relative contents of both α-SMA for contractile VSMC and CD68+ for macrophages in the plaque were significantly lower in L2AKO mice.

Overall, reduced CMA activity associates with many aspects of more severe atherosclerotic pathology supporting an anti-atherosclerotic protective function for CMA.

### Example 10. Metabolic Dysfunction in CMA-Deficient Mice

To determine the basis for the protective effect of CMA against atherosclerosis, and because of the previously described regulation of hepatic glucose and lipid metabolism by CMA, we evaluated metabolic parameters shown to be major risks factors for CVD. We found that L2AKO mice gained 50% more body weight than the WT group during the 12 weeks of WD, mostly due to a higher fat mass content. Indirect calorimetry revealed that the increased adiposity of L2AKO mice did not originate from higher food consumption, but it could be explained by reduced energy expenditure and less physical activity. The decrease in respiratory exchange ratio (RER)—6 indicative of lipid use as energy—observed in WT mice on WD was significantly more pronounced in L2AKO mice, suggesting impaired carbohydrate utilization in these mice. Indeed, L2AKO mice showed marked hyperinsulinemia and increased insulin resistance, typical risk factors for CVD. Circulating levels of the prothrombotic and pro-fibrotic cytokine plasminogen activator inhibitor type 1 (PAI-1) were also significantly higher in L2AKO mice. These findings support that loss of CMA accentuates the systemic derangements in metabolism and coagulation imposed by the WD, thus rendering organisms more prone to atherosclerosis. CMA blockage promotes VSMC dedifferentiation. Whereas circulating cholesterol levels in WT mice show the previously described correlation with different plaque properties, such correlations are lost in L2AKO mice. This suggests that factors other than systemic metabolic changes also contribute to the higher vulnerability of L2AKO mice to atherosclerosis. This motivated us to investigate whether local changes of CMA in the vasculature could contribute to disease progression.

We first examined CMA in primary cultured VSMC exposed to a physiological lipid challenge (LDL loading) and found a dose-dependent upregulation of CMA followed by a gradual decrease once toxic concentrations of LDL are reached. Exposure of L2AKO VSMC to fluorescent LDL (diLDL) resulted in higher intracellular lipid accumulation (FIG. 15A) and reduced cellular viability as LDL concentrations increased (FIG. 15B). This higher susceptibility to lipotoxicity can be primarily attributed to the loss of CMA, since other types of autophagy (i.e., macroautophagy) were fully functional in these cells. Comparative analysis of the transcriptional profile of WT and L2AKO VSMC revealed marked differences under basal conditions and an inadequate transcriptional response after exposure to LDL in L2AKO cells (FIG. 15C). Under basal conditions L2AKO VSMC exhibited loss of ACTA2, an activated-macrophage like gene profile and acquisition of recently identified markers of modified, dedifferentiated VSMC (8) (FIG. 15C). These findings are in line with loss of ACTA2+ contractile SMCs in the plaques in vivo. Gene set enrichment analysis (with STRING database) further showed upregulation of nodes related to cell migration, proliferation, differentiation, and response to lipids.

Loading with LDL induced changes in genes related to lipid metabolism in both genotypes, but we identified quantitative differences in this response. Thus, using Ingenuity Pathway Analysis (IPA), we found that L2AKO cells have a defective response to the lipid challenge with reduced upregulation of genes involved in the cholesterol pathway and display cholesterol as one of the top molecules upregulated in these cells. The immune component of the response of VSMC to lipids is also different in L2AKO cells. While WT cells orchestrate a well characterized inflammatory response, the immune response of L2AKO cells is mainly composed of genes related to leucocyte activation and cell migration. Differential gene expression analysis and gene set enrichment upon lipid loading also identified gene nodes unique for L2AKO cells related with cell death and cellular response to stress, including the response to DNA damage, which we experimentally confirmed to be significantly increased in these cells. These findings support that failure to activate CMA in VSMC makes them unable to adapt to the environmental lipid challenge, as previously described also in CMA-deficient hepatocytes.

Analysis of upstream regulators of the group of genes differentially expressed in L2AKO VSMC revealed as the top change a significant (p<3.13×10−46) downregulation of the tumor protein 53 (p53) signaling pathway. Immunoblot against different components of the p53 signaling pathways confirmed markedly reduced levels of p53 protein and of the cyclin-dependent kinase inhibitor 1A (p21) in L2AKO VSMC, whereas cyclin-dependent kinase inhibitor 1B (p27) content was higher in these cells compared with WT. Considering the well-characterized role of p53 as anti-apoptotic molecule in response to lipid challenges, the identified defect in p53 signaling in L2AKO VSMC provides an explanation for their higher death count (FIG. 15B), increased DNA damage and higher proliferation rates upon LDL loading. Furthermore, relevant to this study is the fact that p53 has previously been shown to protect against VSMC dedifferentiation, a transcriptional pattern already noticeable in L2AKO VSMC under basal conditions (FIG. 15C). We also detected that L2AKO VSMC show constitutively higher intracellular content of the pro-inflammatory and damage-danger-associated molecule pattern (DAMP) chaperone high mobility group box protein-1 (HMGB1), known to complex with p53 and to stimulate PAI-1. Even more striking was the augmented release of HMGB1 into the extracellular media in the form of large molecular weight complexes. This continuous release of HMGB1 from L2AKO VSMC in the arterial wall may be one of the major drivers of the local inflammation and calcium deposition observed in the aortas of L2AKO mice and may also contribute to perpetuate dedifferentiation of CMA-defective VSMC. Also, as part of the possible impact of L2AKO VSMC in arterial wall, and in agreement with our in vivo observations (FIG. 14G), we found a marked increase in most of collagen genes previously associated with plaque fibrosis, which further supports VSMC transition into a synthetic phenotype. Our findings in L2AKO VSMC confirm that fully functional CMA is required in their defense against lipotoxicity and to maintain the identity of VSMC by preventing their dedifferentiation into secretory-migratory cells.

### Example 11. Pro-Inflammatory Phenotype of CMA-Defective Macrophages

The presence of macrophages in the plaque and their associated inflammatory phenotype influence plaque fate. Therefore, we next set to investigate the consequences of CMA blockage in macrophage function using in vitro protocols for polarization of bone marrow-derived macrophages (BMDM) to mimic the plaque pro-inflammatory phenotype of these cells (IFNγ+LPS). We found that CMA-defective BMDM, when stimulated with IFNγ+LPS, show a stronger pro-inflammatory profile (higher inducible nitric oxide synthase, iNOS, and cytochrome c oxidase 2 (COX2) levels (FIG. 16A,B) suggesting that CMA may modulate pro-inflammatory polarization of macrophages. Interestingly, although the changes in COX2 levels were in large part due to its transcriptional upregulation, in the case of iNOS the increase was only at the level of protein, in support of changes in protein degradation contributing to the observed elevated cellular iNOS levels (FIG. 16A,B). We did not observe differences in macroautophagic activity between WT and L2AKO BMDM, in support of the observed phenotype being primarily a consequence of loss of CMA.

We aimed to identify the subset of the proteome that, by not undergoing degradation through CMA, could be behind this exacerbated inflammatory phenotype seen in the L2AKO BMDM. Thus, we isolated the pool of lysosomes usually active for CMA, those that contain high levels of luminal HSC70, from WT and L2AKO BMDM, untreated (CTRL) or stimulated with IFNγ+LPS. In half of the cultures, we inhibited lysosomal proteolysis to discriminate proteins undergoing degradation inside lysosomes from lysosomal resident proteins and subjected the samples to comparative quantitative proteomics (21). About 45% of the proteins were constitutive lysosomal components, not degraded in lysosomes in resting or stimulated BMDM. CMA substrates are defined as those proteins undergoing degradation in lysosomes in a LAMP-2A-dependent manner. Stimulation with IFNγ+LPS resulted in an increase of lysosomal protein degradation, mostly of CMA substrates (46% increase in CMA substrates vs. only 15% increase in non-CMA lysosomal substrates; FIG. 4F). In addition, the repertoire of CMA substrates degraded by untreated and stimulated BMDM was largely different, with only 7% coinciding proteins. Data mining using STRING and IPA identified that the top cellular pathways associated with unstimulated macrophages were related with regulation of immune response, cell adhesion molecules and leucocyte activation, besides the expected upregulation of the pro-inflammatory LPS signaling pathway. The 8 IFNγ+LPS treatment induced CMA degradation of nitic oxide synthase along with five other stimulators of NO synthesis, which can explain the higher levels of iNOS in CMA incompetent macrophages upon stimulation (FIG. 16A). CMA substrates in this condition also included proteins involved in immune response, neutrophil degradation and transendothelial migration (including cell adhesion, cellular localization, and interaction with the vascular wall). The in vivo data confirmed these findings since we found that L2AKO mice showed marked monocytosis, mainly derived from a higher number of proinflammatory monocytes (FIG. 16C), and elevated number of T-cells, especially CD4+ T-cells.

Overall, our findings support that CMA contributes to the remodeling of the proteome induced by macrophage stimulation, and that defective CMA in these cells promotes a more proinflammatory phenotype.

1. A method of preventing or slowing advancement of an age-related neurodegenerative disease in a subject in need thereof, comprising
identifying an early symptom or biomarker of the neurodegenerative disease in the subject, and
administering a therapeutically effective amount of a CMA activator to the subject,
wherein the subject is asymptomatic or is in an early symptomatic stage of the age-related neurodegenerative disease,
wherein administering the CMA activator reduces the progression of beta-amyloid and/or tau pathology in the subject, and/or reduces pre-existing beta-amyloid and/or tau pathology in the subject.

2. (canceled)

3. The method of claim 1, the biomarker is beta-amyloid or tau and the method further comprises determining the progression of beta-amyloid and/or tau pathology by positron emission tomography (PET) and/or magnetic resonance (MR) imaging.

4. The method of claim 1, wherein administering the CMA inhibitor reduces gliosis in the brain of the subject, for example as determined by positron emission tomography (PET) and/or magnetic resonance (MR) imaging.

5. The method of claim 1, wherein the subject is suffering from mild cognitive impairment.

6. The method of claim 1, wherein the age-related neurodegenerative disease is Alzheimer's disease (AD), Lewy body dementia, Parkinson's disease (PD), Huntington's disease, Amyotrophic lateral sclerosis (ALS), Frontotemporal dementia (FTD), Spinocerebellar ataxias (SCAs), and Progressive subcortical gliosis.

7. The method of claim 1, wherein the neurodegenerative disease is AD, and the subject does not suffer from dementia.

8. The method of claim 1, wherein the neurodegenerative disease is AD, and the early symptom is memory loss and/or confusion, difficulty concentrating, difficulty completing daily tasks, time and/or place confusion, difficulty with visual images and/or spatial relationships, difficulty conversing, misplacing objects, poor judgment, withdrawal from activities, olfactory dysfunction, changes in mood and personality.

9. The method of claim 1, wherein the neurodegenerative disease is AD, and the biomarker is tau protein (total tau or phosphorylated tau) or beta-amyloid (e.g., Aβ42) in the plasma or cerebrospinal fluid (CSF) of the subject; or
the neurodegenerative disease is PD and the early symptom is slight tremors in the fingers, thumbs, hand or chin; small handwriting (also called micrographia); loss of smell; difficulty sleeping including sudden movements in sleep; difficulty moving or walking; constipation; a soft or low voice; facial masking; impaired memory; dizziness or fainting; or stooping, leaning and/or slouching while standing.

10. (canceled)

11. The method of claim 1, further comprising detecting an increase in neuronal glycolysis after administering the CMA activator.

12. A method of enhancing neuronal, vascular cell, or macrophage proteostasis in a subject in need of treatment for an age-related neurodegenerative disorder, comprising
administering a CMA activator to the subject, and
detecting an increase in neuronal glycolysis after administering the CMA activator wherein administering the CMA activator enhances neuronal proteostasis in the subject and reduces the progression of beta-amyloid and/or tau pathology in the subject, and
the method optionally further comprises determining the progression of beta-amyloid and/or tau pathology by positron emission tomography (PET) and/or magnetic resonance (MR) imaging, or by tau protein (total tau or phosphorylated tau) or beta-amyloid (e.g., Aβ42) in the plasma or cerebrospinal fluid (CSF) of the subject.

13-16. (canceled)

17. A method of maintaining glycolytic activity in neurons of a patient. comprising
administering an amount of a Chaperone Mediated Autophagy (CMA) activator to the patient sufficient to activate CMA in the excitatory and/or inhibitory neurons of the patient; and thereby maintaining glycolytic activity in the patient's neurons.

18. A method of reducing the level of a marker Alzheimer's Dementia (AD) pathology or slowing the increase of a marker of AD pathology in a patient diagnosed as at risk of developing AD or in a patient diagnosed as having AD, comprising
determining a first level of the marker of AD pathology in the patient
administering an amount of a Chaperone Mediated Autophagy (CMA) activator to the patient sufficient to activate CMA in the neurons of the patient daily for a period of at least 3 months, at least 6 months, or at least 12 months; and
determining a second level of the marker of AD pathology in the patient after the administration of the CMA activator;
comparing the first level and the second level of the marker AD pathology; and determining whether the level of the marker of AD pathology has decreased or slowed in the patient; wherein the marker of AD pathology is a phosphorylation of tau, oligomerization of tau, an increase in beta-amyloid, an increase insoluble tau, or an increase in α-synuclein protein.

19-26. (canceled)

27. A method of preventing, reducing, or slowing advancement of atherosclerotic disease in a subject, comprising
identifying an early symptom or biomarker of atherosclerotic disease in the subject, or identifying the presence of an arterial plague or arterial fatty streak in the subject, and
administering a therapeutically effective amount of a chaperone-mediated autophagy (CMA) activator to the subject,
wherein the subject is asymptomatic or is in an early symptomatic stage of the atherosclerotic disease;
wherein a sufficient amount of the CMA activator is administered to reduce the fraction cholesterol and/or triglycerides in the VLDL and LDL fractions of the subject relative to the level of cholesterol and/or triglycerides in the VLDL and LDL fractions prior to administration of the CMA activator.

28. (canceled)

29. The method of claim 27, wherein the biomarker is vascular smooth muscle cell (VSMC) dedifferentiation or C-reactive protein (CRP) level and the advancement of atherosclerotic disease is monitored by angiogram, chest x-ray, CT scan, echocardiogram, electrocardiogram, intravascular ultrasound, exercise stress test, positron emission tomography (PET) and/or magnetic resonance (MR) imaging.

30. The method of claim 27, wherein administering the CMA activator reduces arterial plaque in the subject, relative to the level of arterial plague in the subject prior to administration of the CMA activator.

31. The method of claim 27, wherein the subject has observable arterial plaques or arterial fatty streak but does not have insufficient blood flow.

32-35. (canceled)

36. A method of reducing a C-reactive protein level in a subject identified as at risk of developing atherosclerosis, comprising:
determining a first level C-reactive protein in the patient; and
administering an amount of a Chaperone Mediated Autophagy (CMA) activator to the patient sufficient to activate CMA in the vascular tissue of the patient; wherein the CMA activator is administered at least once daily for a period of at least 6 weeks, at least 3 months, at least 6 months, or at least 12 months;
determining a second level of the marker of C-reactive protein in the patient after the period of administration of the CMA activator; and
comparing the first level and the second level of the C-reactive protein in the subject.

37. A method of reducing the risk of a stroke in a subject who has already experienced at least one stroke, comprising administering a therapeutically effective amount of a CMA activator to the subject.

38-39. (canceled)
